(19) United States
(12) Patent Application Publication
(10) Pub. No.: US 2023/0192772 A1
(43) Pub. Date: Jun. 22, 2023

(54) ANTIMICROBIAL PEPTIDES AND USES THEREOF

(71) Applicants: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris Cedex 13 (FR); UNIVERSITÉ PARIS-SUD 11, Drsay (FR); UNIVERSIDAD NACIONAL AUTÓNOMA DE MÉXICO, México (MX)

(72) Inventors: Claire LACOMBE, Paris (FR); Constance AUVYNET, Paris (FR); Yvonne ROSENSTEIN, Morelos (MX); Emmanuelle SACHON, Vitry-sur-Seine (FR)

(73) Assignees: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris Cedex 13 (FR); UNIVERSITÉ PARIS-SUD 11, Orsay (FR); UNIVERSIDAD NACIONAL AUTÓNOMA DE MÉXICO, México (MX)

(21) Appl. No.: 16/472,447

(22) PCT Filed: Dec. 22, 2017
(86) PCT No.: PCT/FR2017/053835

§ 371 Date: Jun. 21, 2019

(30) Foreign Application Priority Data
Dec. 22, 2016 (FR) 1663231

(51) Int. Cl. C07K 14/00; A61P 29/00

(52) U.S. Cl. CPC C07K 14/001; A61P 29/00

(57) ABSTRACT

Peptides endowed with an anti-inflammatory, anti-angiogenic and/or antimicrobial activity, a medicament or a pharmaceutical composition including such peptides, and the use thereof in the prevention or treatment of inflammatory diseases, and more particularly inflammatory diseases due to or associated with microbial infections, or in the prevention or treatment of angiogenesis-related diseases.

### FIELD OF INVENTION

The invention relates to peptides endowed with an anti-inflammatory, anti-angiogenic and/or antimicrobial activity and the use thereof in the prevention or treatment of inflammatory and/or angiogenesis-related diseases.

### BACKGROUND OF INVENTION

Inflammation or the inflammatory reaction is a response to an attack of exogenous (infectious, injury-related cause) or endogenous (encounter with an “abnormal” or large quantity of an antigen, immunological cause, ischemia-reperfusion syndrome). It is involved in natural immunity in response to a danger signal as well as in the induction of the specific immune response and is hence associated with a very great variety of pathological scenarios (infections, systemic diseases, cancers, thromboembolic conditions). The inflammatory reaction, initially intended to protect the organism against external attacks, may give rise to harmful effects.

Thus, if the inflammation is inappropriate or becomes chronic, it may impair the structure as well as the function of the organ affected and must be treated.

In particular, the skin and mucous membranes are the site of numerous pathologies comprising an inflammatory component. Eczema and psoriasis are examples of dermatosis characterised by an inappropriate, excessive or chronic inflammatory reaction resulting in tissue lesions.

There are two major types of eczema. Contact dermatitis (or contact eczema) is due to sensitisation to haptens (molecules of low molecular weight) from day-to-day or professional life (heavy metals, solvents, etc.), which diffuse through the epidermis in contact with the skin surface, followed by an intense and inappropriate immune reaction according to a delayed cell-mediated hypersensitivity mechanism. Atopic dermatitis (AD or atopic eczema) is a chronic inflammatory disease combining innate skin barrier dysfunction and inflammation involving innate and adaptive immunity, particularly specific T lymphocytes targeted against environmental protein allergens (extrinsic or allergic AD) or against skin autoantigens (intrinsic or non-allergic AD). AD appears to be correlated with Staphylococcus aureus colonisation of the skin (Lin et al. 2007. Clinic Rev Allerg Immunol; 33:167-177). Psoriasis is a chronic inflammatory disease for which the exact cause is not known. A number of factors would appear to be involved in the onset of the disease, in particular genetic and environmental factors. Microorganisms could also be the cause of psoriasis (Munz et al. 2010. Arch Dermatol Res.; 302(7):495-8). On a cellular level, T lymphocytes appear to play a central role. A frequent complication of inflammatory dermatoses is impetiginisation, i.e. secondary infection of initially non-infected skin lesions, most frequently by streptococci or staphylococci.

The treatment of inflammatory dermatoses consists of boosting skin resistance using emollients and reducing inflammation and itching (pruritis) by the topical application of anti-inflammatory agents. Anti-inflammatory agents comprise steroidal anti-inflammatory drugs (cortisol or cortisone derivatives) and non-steroidal anti-inflammatory drugs (e.g. aspirin, ibuprofen), and compounds having an immunosuppressant activity. The anti-inflammatory agents most commonly used on the skin are dermal corticosteroids such as hydrocortisone or betamethasone. There are four categories of dermal corticosteroids distributed according to the intensity of the activity thereof (potency), from class I (very strong activity) to class IV (weak activity). The choice of the level of activity and dosage of the local corticosteroid is made according to the nature of the lesion, the sensitivity and age of the patient, and may require various adjustments during treatment. When used appropriately, the side-effects of dermal corticosteroids range from skin atrophy (reversible) to the onset of skin infections, requiring treatment discontinuation, and including corticosteroid-associated contact dermatitis.

Anti-inflammatory agents are contraindicated when the skin is infected or if it presents with ulcerations (wounds) and are avoided in cases of infectious dermatosis or of secondary infection of pre-existing dermatosis due to the risk of undetectable progression of the infectious disease, masked by an apparent favourable clinical effect. Thus, it is frequently recommended to treat any skin infection prior to the application of dermal corticosteroids, which complicates the treatment further.

There is therefore a need for novel active ingredients suitable for being used effectively for the prevention and/or treatment of inflammatory and/or infectious diseases, in particular inflammatory dermatoses, particularly cases due to or associated with an infection.

Antimicrobial peptides (AMPs) are defence peptides considered as one of the key elements of the innate immune system, which provides the first line of defence of multicellular organisms against pathogens. The interest for these peptides stems particularly from the broad spectrum of activity thereof. The mode of action thereof is based on a rapid permeabilization or fragmentation of the membranes of the microorganisms, unlikely to generate the onset of resistance mechanisms. Antimicrobial peptides have been identified in plants, insects, batrachians and mammals. The skin of batrachians is an important source of antimicrobial peptides and each frog species has its own peptide repertoire generally consisting of 10 to 15 AMPs.

The Hylidae form a family of amphibians comprising approximately 51 genera divided into three subfamilies: Pelodryadinae, Phyllomedusinae, and Hylinae. The Phyllomedusinae subfamily synthesises and secretes a great diversity of AMPS which have been classified into seven families: dermaseptins (17 peptides), phylloseptins (17 peptides), plasticins (6 peptides), dermatoxins (4 peptides), phylloxins (2 peptides), hyposins (5 peptides) and 4 orphan peptides (Amiche et al. 2008. Peptides; 29:2074- 2082).

The inventors discovered that a fraction of the secretions of Pachymedusa dacnicolor, a tree frog of the Phyllomedusinae subfamily, contains three forms of the same peptide which only differ from one another by the deletion of one or two residues from the C-terminus. The long form of this peptide corresponds to the sequence of a putative dermaseptin PD-2-2 identified from frog cDNA (Wechselberger. 1998. Biochimica et Biophysica Acta; 1388:279-283). The international application WO200055337 describes the use of AMPs, of which PD-2-2, for the manufacture of pathogen-resistant transgenic plants. The international application WO2007112069 describes the use of AMPS, of which PD-2-2, for reducing food consumption, particularly of patients subject to obesity.

In this context, the aim of the present invention is that of providing novel peptides endowed with an anti-inflammatory and/or antimicrobial activity, particularly for the treatment of inflammatory diseases, in particular inflammatory diseases due to or associated with an infection.

### SUMMARY

The present invention relates to a peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-Cp is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, SEQ ID NO: 3 or SEQ ID NO: 4.

In one embodiment, the peptide with an anti-inflammatory activity and/or antimicrobial activity has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, SEQ ID NO: 3 or SEQ ID NO: 4.

In one embodiment, the peptide has a sequence selected from the group comprising SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 26, SEQ ID NO: 27, SEQ ID NO: 28 and functional derivatives and pharmaceutically acceptable salts of this peptide.

The invention further relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, or a nucleotide sequence coding for at least one of these peptides, for use as a medicament.

The present invention relates to a medicament comprising a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, or a nucleotide sequence coding for at least one such peptide.

In one embodiment, the medicament comprises a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, or a nucleotide sequence coding for at least one of these peptides.

The invention also relates to a pharmaceutical composition comprising at least one peptide as described above or a nucleotide sequence coding for at least one of these peptides, and one or a plurality of pharmaceutically acceptable excipients.

Another object of the invention is a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use as an anti-inflammatory and/or immunosuppressant agent.

In one embodiment, the peptide has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use as an anti-inflammatory and/or immunosuppressant agent.

One aspect of the invention relates to a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use in the prevention or treatment of an inflammatory disease in a subject.

In one embodiment, the peptide has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use in the prevention or treatment of an inflammatory disease in a subject.

In one embodiment, the peptide, medicament or pharmaceutical composition for use as described above, is characterised in that the inflammatory disease is due to an infection.

One aspect of the invention relates to a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use in the prevention or treatment of an angiogenesis-related disease in a subject.

In one embodiment, the peptide has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a nucleotide sequence coding for at least one of these peptides, a medicament as described above or pharmaceutical composition as described above, for use in the prevention or treatment of an angiogenesis-related disease in a subject.

The present invention further relates to the use of a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A, X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, as an antifungal or antibacterial agent.

The present invention further relates to the use of a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, as an antifungal or antibacterial agent.

The present invention also relates to a kit comprising a peptide, a peptide for use, a medicament, or a pharmaceutical composition as described above, and optionally the instructions for administering the peptide, medicament, or pharmaceutical composition to said subject.

### DEFINITIONS

In the present invention, the terms “peptide”, “oligopeptide”, “polypeptide” or “protein” are used interchangeably and refer to a chain of amino acids linked by peptide bonds, regardless of the number of amino acid residues forming this chain. In the peptide sequences described in this document, the amino acids are represented by the one-letter or three-letter code thereof according to the following nomenclature: C: Cys, cysteine; D: Asp, aspartic acid; E: Glu, glutamic acid; F: Phe, phenylalanine; G: Gly, glycine; H: His, histidine; I: Ile, isoleucine; K: Lys, lysine; L: Leu, leucine; M: Met, methionine; N: Asn, asparagine; P: Pro, proline; Q: Gln, glutamine; R: Arg, arginine; S: Ser, serine; T: Thr, threonine; V: Val, valine; W: Trp, tryptophan and Y: Tyr, tyrosine.

The term “microorganism” or “microbial” as used in this document refers to bacteria, fungi, yeasts, viruses and/or parasites.

The term “microbial infection” as used in this document refers to an infection due to bacteria, fungi, yeasts, viruses and/or parasites.

The term “antimicrobial activity” as used in this document refers to an antibacterial, antiviral, antifungal and/or antiparasitic activity. This activity may be evaluated by means of the measurement of various parameters such as the IC₅₀, the MIC or the MBC. The “IC₅₀” or “median inhibitory concentration” corresponds to the concentration of a compound required to reduce the in vitro growth of a microorganism population by 50%. The “MIC” or “minimum inhibitory concentration” corresponds to the minimum concentration of compound suitable for inhibiting microbial growth completely, after 18 hours of incubation, in general at 37° C., in the presence of said compound. The “MBC” or “minimum bactericidal concentration” is the minimum concentration of compound suitable for destroying 99.9% of the microorganisms after 18 to 24 hours of contact with said compound.

The term “median lethal concentration” or “LC₅₀” as used in this document refers to the concentration of compound causing the death of 50% of a cell population. The LC₅₀ is a quantitative indicator of the toxicity of a compound. The LC₅₀ is particularly used in this document to assess the cytotoxicity of AMPs, in particular on immune and erythrocyte cells. In one embodiment, the LC₅₀ corresponds to the concentration of peptide inducing the death of 50% of immune cells. In a further embodiment, the LC₅₀ corresponds to the concentration of peptide inducing the death of 50% of erythrocyte cells.

The term “anti-inflammatory activity” as used in this document refers to an activity aimed at inhibiting or reducing inflammation. Mention may be made by way of example of erythema, oedemas. Inflammation, in medical terms, is defined as the combination of the following four phenomena: swelling, redness, heat and pain.

The term “immunosuppressant activity” as used in this document refers to an activity aimed at attenuating or suppressing the immune reactions of the organism.

The term “pharmaceutically acceptable” refers only to the ingredients of a pharmaceutical composition that are mutually compatible and not harmful for the patient. In one embodiment, a pharmaceutically acceptable excipient does not produce any side-effect, allergic effect or other adverse reaction when administered to an animal, preferably a human. For human administration, the preparations must meet the standard sterility, pyrogenicity, general safety and purity requirements as stipulated by regulatory bodies, such as for example the FDA or EMA.

The term “subject” refers to an animal, including a human being. According to the present invention, a subject may be a patient, namely a person receiving medical care, undergoing or having undergone a medical treatment, or monitored for disease progression.

The term “treat” or “treatment” refers both to a therapeutic treatment and to prophylactic or preventive measures, wherein the objective is to prevent or slow down (reduce) the targeted pathological condition, or disorder. Those needing treatment include those already suffering from the disorder and those who are predisposed or susceptible to having it, and also those in whom the disorder is to be prevented. A subject or a mammal is “treated” successfully if, after having received a therapeutic dose of the peptide according to the present invention, the patient shows an observable and/or measurable reduction or an absence of one or more of the following elements: reduction in the number of pathogenic cells, reduction in the total percentage of pathogenic cells, and/or a certain relief of one or more of the symptoms associated with the targeted disease, reduced morbidity and mortality, and an improvement of quality of life. The above parameters for assessing the success of the treatment and the improvement of the disease status are readily measurable by means of routine procedures known to the practitioner.

The term “therapeutically effective dose” refers to the dose of therapeutic agent required and sufficient to slow down or stop the progression, worsening or deterioration or one of a plurality of symptoms of the disease or conditions; relieve the symptoms of the disease or condition; cure the disease or condition. In one embodiment, the condition according to the invention is an inflammatory disease, a microbial disease, or an inflammatory disease with an infectious component. In one embodiment, the condition according to the invention is an angiogenesis-related disease.

### DETAILED DESCRIPTION

The invention firstly relates to a peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence

ALWKTLLKKVGKVAGKAVLNAVTNMANQNEQOH (SEQ ID NO: 2)

,

ALWKTLLKKVGKVAGKAVLNAVTNMANQNE-OH (SEQ ID NO: 3)

or

ALWKTLLKKVGKVAGKAVLNAVTNMANQN-OH (SEQ ID NO: 4)

.

In one embodiment, the peptide according to the invention does not comprise SEQ ID NO: 2.

In one embodiment, the peptide according to the invention does not comprise SEQ ID NO: 3.

In one embodiment, the peptide according to the invention does not comprise SEQ ID NO: 4.

According to one embodiment of the invention, the peptide according to the invention is not a peptide of sequence

MALVKKSLFLVLFLGLVSLSICEEKRENEDEEEQEDDEQSEEKRALWKTL
LKKVGKVAGKAVLNAVTNMANQNEQ (SEQ ID NO: 36).

According to one embodiment of the invention, the peptide according to the invention does not comprise the sequence SEQ ID NO: 36.

In one embodiment, φ is X₇-L-N-A-V-T-N-X₈-A-N-Q-N-X₉-X₁₀ (SEQ ID NO: 32) and/or X₉ and X₁₀ represent no residue. In one embodiment, φ is X₇-L-N-A-V-T-N-X₈-A-N-Q-N-X₉-X₁₀ (SEQ ID NO: 32) and X₉ and X₁₀ represent no residue.

In one embodiment, the peptide with an anti-inflammatory activity and/or antimicrobial activity has a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2, SEQ ID NO: 3 or SEQ ID NO: 4.

In one embodiment, the peptide with an anti-inflammatory activity and/or antimicrobial activity has a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2, SEQ ID NO: 3 or SEQ ID NO: 4.

In one embodiment, the peptide with an anti-inflammatory activity and/or antimicrobial activity has a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2, SEQ ID NO: 3 or SEQ ID NO: 4.

In one embodiment, the peptide with an anti-inflammatory activity and/or antimicrobial activity has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, SEQ ID NO: 3 or SEQ ID NO: 4.

In a preferred embodiment of the invention, the peptide according to the invention is endowed with an anti-inflammatory activity, an immunosuppressant activity, an anti-angiogenic activity and/or an antimicrobial activity.

In one embodiment of the invention, the peptide according to the invention is endowed with an antimicrobial activity. In a further embodiment of the invention, the peptide according to the invention is endowed with an anti-inflammatory activity. In a further embodiment of the invention, the peptide according to the invention is endowed with an immunosuppressant effect. In a further embodiment of the invention, the peptide according to the invention is endowed with an anti-angiogenic effect.

In a preferred embodiment of the invention, the peptide according to the invention is endowed with an anti-inflammatory activity and an antimicrobial activity. In a preferred embodiment of the invention, the peptide according to the invention is endowed with an anti-inflammatory activity and an immunosuppressant activity. In a preferred embodiment of the invention, the peptide according to the invention is endowed with an anti-inflammatory activity and an anti-angiogenic activity.

In a preferred embodiment of the invention, the peptide according to the invention is endowed with an anti-inflammatory activity, an immunosuppressant activity and an antimicrobial activity. In a preferred embodiment of the invention, the peptide according to the invention is endowed with an anti-inflammatory activity, an anti-angiogenic activity and an antimicrobial activity. In a preferred embodiment of the invention, the peptide according to the invention is endowed with an anti-inflammatory activity, an immunosuppressant activity and an anti-angiogenic activity. In a preferred embodiment of the invention, the peptide according to the invention is endowed with an immunosuppressant activity, an anti-angiogenic activity and an antimicrobial activity.

In a preferred embodiment of the invention, the peptide according to the invention is endowed with an anti-inflammatory activity, an immunosuppressant activity, an anti-angiogenic activity and an antimicrobial activity.

In a preferred embodiment of the invention, the residue X₁ is W in SEQ ID NOs 1 and 29 to 32, the residue X₂ is K in SEQ ID NOs 1 and 29 to 32, the residue X₃ is L in SEQ ID NOs 1 and 29 to 32, the residue X₄ is K or A in SEQ ID NOs 1 and 29 to 32, the residue X₅ is V in the sequences SEQ ID NOs 1 and 29 to 32, the residue X₆ is A in SEQ ID NOs 1 and 29 to 32, and/or the residue X₇ is V in SEQ ID NOs 1, 29, 31 and 32. In a preferred embodiment of the invention, the residue X₉ represents the E residue and the residue X₁₀ represents the Q residue in SEQ ID NO 1.

In a further preferred embodiment of the invention, the residue X₉ represents no amino acid in SEQ ID NO 1. In a preferred embodiment of the invention, the residue X₁₀ represents no amino acid in SEQ ID NO 1. In a preferred embodiment of the invention, the residue X₉ is E and the residue X₁₀ is absent in SEQ ID NO 1. In a preferred embodiment of the invention, the residue X₉ represents the E residue and the residue X₁₀ represents no amino acid in SEQ ID NO 1.

In a further preferred embodiment of the invention, the residues X₉ and X₁₀ represent no amino acid. Thus, in a preferred embodiment of the invention, the peptide according to the invention has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N (SEQ ID NO: 25), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue; and
functional derivatives and pharmaceutically acceptable salts of said peptide, wherein the peptide is not a peptide of sequence SEQ ID NO: 2; SEQ ID NO: 3 or SEQ ID NO: 4.

According to one embodiment, the peptide has a size between 17 and 100 amino acids, preferably between 17 and 50, between 17 and 45, between 17 and 40, between 17 and 35, or between 17 and 30 amino acids. According to a further embodiment, the peptide has a size between 17 and 34, between 17 and 33, between 17 and 32, or between 17 and 31 amino acids. According to one embodiment, the peptide has a size of 17 amino acids.

According to one embodiment, the peptide has a size between 21 and 100 amino acids, preferably between 21 and 50, between 21 and 45, between 21 and 40, between 21 and 35, or between 21 and 30 amino acids. According to a further embodiment, the peptide has a size between 21 and 34, between 21 and 33, between 21 and 32, or between 21 and 31 amino acids. According to one embodiment, the peptide has a size of 21 amino acids.

According to one embodiment, the peptide has a size between 26 and 100 amino acids, preferably between 26 and 50, between 26 and 45, between 26 and 40, between 26 and 35, or between 26 and 30 amino acids. According to a further embodiment, the peptide has a size between 26 and 34, between 26 and 33, between 26 and 32, or between 26 and 31 amino acids. According to one embodiment, the peptide has a size of 26 amino acids.

According to one embodiment, the peptide has a size between 29 and 100 amino acids, preferably between 29 and 50, between 29 and 45, between 29 and 40, between 29 and 35, or between 29 and 30 amino acids. According to a further embodiment, the peptide has a size between 29 and 34, between 29 and 33, between 29 and 32, or between 29 and 31 amino acids.

According to a further embodiment, the peptide has a size of 17, 18, 19, 20, 21, 22, 23, 24, 25, 26, 27, 28, 29, 30, 31, 32, 33, 34 or 35 amino acids. In a preferred embodiment, the peptide has a size of 29 amino acids.

The amino acids forming the peptide according to the invention may be of L or D configuration, preferably of L configuration. In one embodiment of the invention, the peptide according to the invention may contain a combination of amino acids of L configuration and of D configuration.

The peptide according to the invention may have a post-translational modification and/or a chemical modification in particular a glycosylation, an amidation, an esterification, an acylation, an acetylation or a methylation.

Indeed, so as to increase the bioavailability of the peptide by enhancing the peptidase resistance thereof, protecting groups may be added to the C- and/or N-termini. For example, the protecting group at the N-terminus may be an acylation or an acetylation and the protecting group at the C-terminus may be an amidation or an esterification. The action of proteases may also be impeded with the use of amino acids of D configuration, peptide cyclisation by the formation of disulphide bridges, lactam rings or bonds between the N- and C-termini. The peptide according to the invention may also comprise pseudopeptide bonds replacing “conventional” CONH peptide bonds and conferring an increased peptidase resistance, such as CHOH—CH₂, NHCO, CH₂—O, CH₂CH₂, CO—CH₂, N—N, CH, CH₂NH, and CH₂—S. Preferably, the peptide according to the invention has an amidation of the C-terminus thereof.

According to one embodiment, the peptide according to the present invention consists of or comprises a sequence selected from the following group:

ALWKTLLKKVGKVAGKAVLNAVTNMANQNEQ-NH2 (SEQ ID NO: 5);

ALWKTLLKKVGKVAGKAVLNAVTNMANQNE-NH2 (SEQ ID NO: 6);

ALWKTLLKKVGKVAGKAVLNAVTNMANQN-NH2 (SEQ ID NO: 7);

ALWKTLLKKVGKVAGKAVLNAVTNMA-NH2 (SEQ ID NO: 26);

ALWKTLLKKVGKVAGKAVLNA-NH2 (SEQ ID NO: 27);

ALWKTLLKKVGKVAGKA-NH2 (SEQ ID NO: 28);

ALWKTLLKKVGKVKGKAVLNAVTNMANQN (SEQ ID NO: 8);

ALWKTLLKKVGKVAGKAPLNAVTNMANQN (SEQ ID NO: 9);

ALWKTLLKKVGKVDGKAVLNAVTNMANQN (SEQ ID NO: 10);

ALWKTLLKKVGAVAGKAVLNAVTNMANQN (SEQ ID NO: 11);

ALWKTLLKKVGPVAGKAVLNAVTNMANQN (SEQ ID NO: 12);

ALWDTLLKKVGKVAGKAVLNAVTNMANQN (SEQ ID NO: 13);

ALWKTLAKKVGKVAGKAVLNAVTNMANQN (SEQ ID NO: 14);

ALAKTLLKKVGKVAGKAVLNAVTNMANQN (SEQ ID NO: 15);

ALWKTLLKKVGKVAGKAVLNAVTNAANQN (SEQ ID NO: 16);

ALWKTLLKKVGKAAGKAVLNAVTNMANQN (SEQ ID NO: 17);

dAdLdWdKdTdLdLdKdKdVdGdKdVdAdGdKdAdVdLdNdAdVdTdNdM
dAdNdQdNdEdQ
(SEQ ID NO: 18);

dAdLdWdKdTdLdLdKddKdVdGdKdVdAdGdKdAdVdLdNdAdVdTdNd
MdAdNdQdN
(SEQ ID NO: 19);

dQdEdNdQdNdAdMdNdTdVdAdNdLdVdAdKdGdAdVdKdGdVdKdKdL
dLdTdKdWdLdA
(SEQ ID NO: 20); and

dNdQdNdAdMdNdTdVdAdNdLdVdAdKdGdAdVdKdGdVdKdKdLdLdT
dKdWdLdA (SEQ ID NO: 21),

wherein the residues preceded by the letter “d” are amino acids of D configuration, and functional derivatives and pharmaceutically acceptable salts of said peptide.

In one embodiment, the peptide has a sequence comprising or consisting of the sequence SEQ ID NO: 5. In one embodiment, the peptide has a sequence comprising or consisting of the sequence SEQ ID NO: 6. In one embodiment, the peptide has a sequence comprising or consisting of the sequence SEQ ID NO: 7. In one embodiment, the peptide has a sequence comprising or consisting of the sequence SEQ ID NO: 8.

The invention also covers functional derivatives of a peptide according to the invention as described above. The term “functional derivative” as used in this document, refers to peptides having substantially the same amino acid sequence, substantially the same helical structure and substantially the same anti-inflammatory, anti-angiogenic and/or antimicrobial activity. Preferentially, the term “functional derivative” as used in this document, refers to peptides having substantially the same amino acid sequence, substantially the same helical structure and substantially the same anti-inflammatory and/or antimicrobial activity. These functional derivatives may be resistant to proteolysis by one or a plurality of chemical modification(s) or substantially homologous by one or a plurality of conservative replacement(s). These functional derivatives may be, for example, inverso peptides, retro peptides, retro-inverso peptides and peptides wherein the side chain of one or a plurality of the amino acids is substituted by groups that do not modify the anti-inflammatory and/or antimicrobial activity of the peptide according to the invention.

The invention also covers the pharmaceutically acceptable salts of a peptide according to the invention. The pharmaceutically acceptable salts may be, for example, salts with pharmaceutically acceptable mineral acids such as hydrochloric acid, hydrobromic acid, sulphuric acid and phosphoric acid; salts with pharmaceutically acceptable organic acids such as acetic acid, citric acid, maleic acid, malic acid, succinic acid, ascorbic acid and tartaric acid; salts with pharmaceutically acceptable mineral bases such as sodium, potassium, calcium, magnesium or ammonium salts; or salts with organic bases that have a salifiable nitrogen, commonly used in the pharmaceutical art. The methods for preparing these salts are well-known to those skilled in the art.

The peptide according to the invention may be obtained by conventional chemical synthesis (solid phase or liquid homogeneous phase) or by enzymatic synthesis (Kullman et al. 1987. Enzymatic peptide synthesis, CRC Press, Florida). It may also be obtained using the method consisting of culturing a host cell, as described hereinafter, comprising a transgene coding for the peptide and expressing said peptide, and extracting said peptide from these host cells or from the culture medium wherein the peptide has been secreted.

According to one embodiment, the peptide is an isolated peptide. In one embodiment, the peptide is a recombinant peptide.

The invention secondly relates to a nucleic acid coding for a peptide according to the first subject matter of the invention. According to the invention, “nucleic acid” denotes any DNA-or RNA-based molecule. This may consist of synthetic or semi-synthetic, recombinant molecules, optionally amplified or cloned in vectors, chemically modified, comprising non-natural bases or modified nucleotides comprising for example a modified bond, a modified puric or pyrimidic base, or a modified sugar. The nucleic acid according to the invention may be in single-stranded or double-stranded DNA and/or RNA form. According to a preferred embodiment, the nucleic acid is an isolated DNA molecule, synthesised using recombinant techniques well-known to those skilled in the art. The nucleic acid according to the invention may be deduced from the sequence of the peptide according to the invention and the use of the codons may be adapted according to the host cell wherein the nucleic acid is to be transcribed. These steps may be carried out according to methods well-known to those skilled in the art and some whereof are described in the reference manual of Sambrook et al. (Sambrook et al. 2001. In Vitro Cell. Dev. Biol.; 28P:97-105).

In one embodiment, the nucleic acid is the nucleic acid coding for an amino acid sequence selected from the group comprising or consisting of SEQ ID NO: 1, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 26, SEQ ID NO: 27, and SEQ ID NO: 28.

In one embodiment, the nucleic acid is selected from the group comprising or consisting of the sequences:

GCG CTG TGG AAA ACC TTA CTG AAG AAA GTG GGC AAG GTT GCG GGT AAA
GCC GTG CTG AAC GCA GTT ACC AAT ATG GCG AAC CAG AAT (SEQ ID NO: 22)

coding for the peptides of sequence SEQ ID NO: 7 and 8;

GCG CTG TGG AAA ACC TTA CTG AAG AAA GTG GGC AAG GTT GCG GGT AAA
GCC GTG CTG AAC GCA GTT ACC AAT ATG GCG AAC CAG AAT GAA (SEQ ID NO:23)

coding for the peptides of sequence SEQ ID NO: 3 and 6; and

GCG CTG TGG AAA ACC TTA CTG AAG AAA GTG GGC AAG GTT GCG GGT AAA
GCC GTG CTG AAC GCA GTT ACC AAT ATG GCG AAC CAG AAT GAA CAA (SEQ ID NO: 24)

coding for the peptides of sequence SEQ ID NO: 2 and 5, or any sequence having at least 75%, preferably at least 80, 85, 90, 95, 96, 97, 98 or 99% identity with one of the sequences SEQ ID NOs: 22-24.

According to the present invention, the term “identity”, when used in a relation between the sequences of two or a plurality of nucleotide sequences, refers to the degree of relationship between these nucleotide sequences, as determined by the number of matches between chains of two or more bases. According to the invention, the “identity” thus corresponds to a percentage of identity between two (or more than 2) sequences. This percentage is defined as the number of positions for which the bases are identical when the sequences are aligned optimally, divided by the total number of bases of the smaller of the two sequences. The differences between the sequences may be distributed at random and along the entire lengths thereof.

Two sequences are said to be optimally aligned when the identity percentage is maximum. Moreover, as will be obvious to those skilled in the art, it may be necessary to use additions of “gaps” so as to obtain an optimal alignment between the two sequences. The identity percentage between two nucleic acid sequences may therefore be determined by comparing these two optimally aligned sequences wherein the nucleic acid sequence to be compared may comprise additions or deletions with respect to the reference sequence for an optimal alignment between these two sequences. The identity percentage is then calculated by determining the number of identical positions for which the nucleotide is identical between the two sequences, by dividing this number of identical positions by the total number of positions in the comparison window and by multiplying the result obtained by 100 to obtain the identity percentage between these two sequences.

Preferentially, the methods for determining the identity are devised to give the greatest possible concordance between the compared sequences.

The identity percentage may be determined by a particular mathematical model or by a computer program (generally referred to as “algorithm”). Methods for computing the identity between nucleotide sequences are well-known to those skilled in the art. Non-limiting examples of such methods include those described in the following documents: Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et αl., 1988. SIAM J. Appl. Math. 48(5): 1073-1082.

Methods for determining the identity have been described in open-access computer programs. Preferred examples of methods using computer programs include, without being limited thereto, GCG software, including GAP (Devereux et αl., 1984. Nucl. Acid. Res. 12(1 Pt 1):387-395; Genetics Computer Group, University of Wisconsin Biotechnology Center, Madison, WI), BLASTP, BLASTN, and FASTA (Altschul et αl., 1990. J. Mol. Biol. 215(3):403-410). The BLASTX program is available from the National Center for Biotechnology Information (NCBI) and other sources (BLAST Manual, Altschul et αl. NCB/NLM/NIH Bethesda, Md. 20894; Altschul et αl., 1990. J. Mol. Biol. 215(3):403-410). The Smith-Waterman algorithm, which is well-known to those skilled in the art, may also be used to determine the identity percentage between two sequences.

The invention further relates to a vector or a plasmid wherein a nucleic acid according to the invention is associated with suitable elements for controlling transcription (in particular a promoter, an amplifier and optionally a terminator) and, optionally translation.

In one embodiment, the vector is a recombinant vector. In one embodiment, a nucleic acid according to the invention has been inserted into the vector. In one embodiment, the vector may be a cloning vector or an expression vector.

The invention also relates to a host cell expressing a peptide according to the invention, constitutively or transiently. In one embodiment, the host cell has been transformed by at least one of the nucleic acids according to the invention.

As used herein, the term “transformation” signifies the introduction of a gene, or of an RNA or DNA sequence (including plasmids and viral vectors) that is “extraneous” (i.e. extrinsic or extracellular) into a host cell, such that the host cell expresses the gene or the sequence introduced to produce the desired substance, typically a protein coded by the gene or the sequence. A host cell that receives and expresses the DNA or RNA introduced has been “transformed”.

The construction of the vectors according to the invention and the transformation of the host cells may be carried out using conventional molecular biology techniques.

The invention also relates to a peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2; or a nucleotide sequence coding for at least one of these peptides, for use as a medicament.

In one embodiment, the peptide according to the invention does not comprise SEQ ID NO: 2.

In one embodiment, the peptide for use as a medicament has a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide for use as a medicament has a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide for use as a medicament has a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide for use as a medicament has a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, preferably a peptide having a sequence of 29 to 100 amino acids comprising the sequence SEQ ID NO: 3, 4, 5, 6, 7 or 8; or a nucleotide sequence coding for at least one of these peptides.

In a preferred embodiment of the invention, the residue X₁ is W in SEQ ID NOs 1 and 29 to 32, the residue X₂ is K in SEQ ID NOs 1 and 29 to 32, the residue X₃ is L in SEQ ID NOs 1 and 29 to 32, the residue X₄ is K or A in SEQ ID NOs 1 and 29 to 32, the residue X₅ is V in SEQ ID NOs 1 and 29 to 32, X₆ is the A residue in SEQ ID NOs 1 and 29 to 32, and/or the residue X₇ is V in SEQ ID NOs 1, 29, 31 and 32. In a preferred embodiment of the invention, the residue X₉ represents the E residue and the residue X₁₀ represents the Q residue in SEQ ID NO 1.

In a further preferred embodiment of the invention, the residue X₉ represents no amino acid in SEQ ID NO 1. In a preferred embodiment of the invention, the residue X₁₀ represents no amino acid in SEQ ID NO 1. In a preferred embodiment of the invention, the residue X₉ is E and the residue X₁₀ is absent in SEQ ID NO 1. In a preferred embodiment of the invention, the residue X₉ represents the E residue and the residue X₁₀ represents no amino acid in SEQ ID NO 1.

In a further preferred embodiment of the invention, the residues X₉ and X₁₀ represent no amino acid.

The invention also relates to a medicament comprising at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide according to the invention does not comprise SEQ ID NO: 2.

In one embodiment, the medicament comprises at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2.

In one embodiment, the medicament comprises at least one peptide with an anti-inflammatory activity and/or antimicrobial activity having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the medicament comprises at least one peptide with an anti-inflammatory activity and/or antimicrobial activity having a sequence of 26 to 100 amino acids comprising the sequence A-L-X₁-X₂-T-L-X₃-K-K-V-G-X₄-V-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the medicament comprises at least one peptide with an anti-inflammatory and/or antimicrobial activity having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, preferably a peptide having a sequence of 29 to 100 amino acids comprising the sequence SEQ ID NO: 3, 4, 5, 6, 7 or 8; or a nucleotide sequence coding for at least one of these peptides.

The present invention also relates to a composition comprising at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide according to the invention does not comprise SEQ ID NO: 2.

In one embodiment, the composition comprises at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide according to the invention does not comprise SEQ ID NO: 2.

In one embodiment, the composition comprises at least one peptide with an anti-inflammatory activity and/or antimicrobial activity having a sequence of 21 to 100 amino acids comprising the sequence A-L-X₁-X₂-T-L-X₃-K-K-V-G-X₄-X₅-X₆-G-K-A-X₆-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the peptide according to the invention does not comprise SEQ ID NO: 2.

In one embodiment, the composition comprises at least one peptide with an anti-inflammatory activity and/or antimicrobial activity having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2; or a nucleotide sequence coding for at least one of these peptides.

In one embodiment, the composition comprises at least one peptide comprising a peptide with an anti-inflammatory and/or antimicrobial activity having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X_{IO} (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, preferably a peptide having a sequence of 29 to 100 amino acids comprising the sequence SEQ ID NO: 3, 4, 5, 6, 7 or 8; or a nucleotide sequence coding for at least one of these peptides.

The invention also relates to a pharmaceutical composition comprising at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-Cp is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, or a nucleotide sequence coding for at least one of these peptides, and one or a plurality of pharmaceutically acceptable excipients.

In one embodiment, the peptide according to the invention does not comprise SEQ ID NO: 2.

In one embodiment, the composition comprises at least one peptide with an anti-inflammatory activity and/or antimicrobial activity having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2, or a nucleotide sequence coding for at least one of these peptides, and one or a plurality of pharmaceutically acceptable excipients.

In one embodiment, the pharmaceutical composition comprises at least one peptide with an anti-inflammatory activity and/or antimicrobial activity having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2, or a nucleotide sequence coding for at least one of these peptides, and one or a plurality of pharmaceutically acceptable excipients.

In one embodiment, the pharmaceutical composition comprises at least one peptide with an anti-inflammatory activity and/or antimicrobial activity having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2, or a nucleotide sequence coding for at least one of these peptides, and one or a plurality of pharmaceutically acceptable excipients.

In one embodiment, the pharmaceutical composition comprises at least one peptide comprising a peptide with an anti-inflammatory and/or antimicrobial activity having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, with the exception of the peptide of sequence SEQ ID NO: 2, preferably a peptide having a sequence of 29 to 100 amino acids comprising the sequence SEQ ID NO: 3, 4, 5, 6, 7 or 8, or a nucleotide sequence coding for at least one of these peptides, and one or a plurality of pharmaceutically acceptable excipients.

The invention further relates to a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₇-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for use as an anti-inflammatory, immunosuppressant and/or anti-angiogenic agent.

In one embodiment, the peptide having a sequence of 17 to 100 amino acids comprising the sequence SEQ ID NO: 29 and functional derivatives and pharmaceutically acceptable salts of said peptide, is for use as an anti-inflammatory and/or immunosuppressant agent.

In one embodiment, the peptide having a sequence of 17 to 100 amino acids comprising the sequence SEQ ID NO: 29 and functional derivatives and pharmaceutically acceptable salts of said peptide, is for use as an anti-inflammatory and/or anti-angiogenic agent.

In one embodiment, the invention relates to a peptide having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, for use as an anti-inflammatory, immunosuppressant and/or anti-angiogenic agent.

In one embodiment, the invention relates to a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, for use as an anti-inflammatory, immunosuppressant and/or anti-angiogenic agent.

In one embodiment, the invention relates to a peptide having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, for use as an anti-inflammatory, immunosuppressant and/or anti-angiogenic agent.

In one embodiment, the invention relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for use as an anti-inflammatory, immunosuppressant and/or anti-angiogenic agent.

In a preferred embodiment of the invention, the residue X₁ is W in SEQ ID NOs 1 and 29 to 32, the residue X₂ is K in SEQ ID NOs 1 and 29 to 32, the residue X₃ is L in SEQ ID NOs 1 and 29 to 32, the residue X₄ is K or A in SEQ ID NOs 1 and 29 to 32, the residue X₅ is V in SEQ ID NOs 1 and 29 to 32, the residue X₆ is A in SEQ ID NOs 1 and 29 to 32, and/or the residue X₇ is V in SEQ ID NOs 1, 29, 31 and 32. In a preferred embodiment of the invention, the residue X₉ represents the E residue and the residue X₁₀ represents the Q residue in SEQ ID NO 1.

In a further preferred embodiment of the invention, the residue X₈ represents no amino acid in SEQ ID NO 1. In a preferred embodiment of the invention, the residue X₁₀ represents no amino acid in SEQ ID NO 1. In a preferred embodiment of the invention, the residue X₉ is E and the residue X₁₀ is absent in SEQ ID NO 1. In a preferred embodiment of the invention, the residue X₉ represents the E residue and the residue X₁₀ represents no amino acid in SEQ ID NO 1.

In a further preferred embodiment of the invention, the residues X₉ and X₁₀ represent no amino acid.

In a preferred embodiment of the invention, the peptide for use as an anti-inflammatory, immunosuppressant and/or anti-angiogenic agent, is a peptide comprising a sequence selected from the group comprising or consisting of the sequences SEQ ID NOs: 2, 3, 4, 5, 6, 7 or 8.

In a preferred embodiment of the invention, the peptide for use as an anti-inflammatory and/or anti-angiogenic agent, is a peptide comprising a sequence selected from the group comprising or consisting of the sequences SEQ ID NOs: 2, 3, 4, 5, 6, 7 or 8.

In one embodiment, the peptide as described above is useful as an immunosuppressant agent. In a preferred embodiment of the invention, the peptide as described above is used as an immunosuppressant agent.

In another embodiment, the peptide as described above is useful as an anti-inflammatory agent. In a preferred embodiment of the invention, the peptide as described above is used as an anti-inflammatory agent.

In another embodiment, the peptide as described above is useful as an anti-angiogenic agent. In a preferred embodiment of the invention, the peptide as described above is used as an anti-angiogenic agent.

In another embodiment, the peptide as described above is useful as an anti-inflammatory, immunosuppressant and anti-angiogenic agent, preferably as an anti-inflammatory and immunosuppressant agent. In a preferred embodiment of the invention, the peptide as described above is used as an anti-inflammatory, immunosuppressant and anti-angiogenic agent, preferably as an anti-inflammatory and immunosuppressant agent.

In one embodiment, the invention relates to the peptide as described above for use in the prevention or treatment of an inflammatory disease, preferably a peptide having a sequence of 29 to 100 amino acids comprising SEQ ID NO: 1, preferably comprising a sequence selected from the group comprising or consisting of the sequences SEQ ID NOs: 2, 3, 4, 5, 6, 7 or 8.

The term “inflammatory disease” as used in this document refers to any disease wherein an inflammatory reaction has pathological consequences for the organism. Inflammatory diseases affect organs or tissues such as the digestive system, nervous system, skin, mucous membranes or joints, inter alia. Acute inflammatory disease according to the invention include for example septicaemia, septic shock. Chronic inflammatory diseases according to the invention include, but are not limited to, septicaemia, septic shock, auto-immune diseases (such as lupus or rheumatoid arthritis), inflammatory heart diseases (carditis, and particularly endocarditis, pericarditis, myocarditis, in particular endocarditis of infectious origin, such as cases induced by Staphylococcus aureus), graft rejection, injuries, inflammatory joint diseases (particularly the various types of arthritis), inflammatory gastrointestinal tract diseases (particularly colitis, enteritis, gastritis, gastro-enteritis, chronic inflammatory bowel diseases (CIBD) such as Crohn’s disease and haemorrhagic recto-colitis (HRC)), inflammatory skin diseases (particularly eczema, contact dermatitis, psoriasis, rosacea, dermatosis), inflammatory respiratory tract diseases (particularly asthma, chronic bronchitis), inflammatory eye diseases (particularly conjunctivitis, episcleritis, scleritis, keratitis, uveitis, blepharitis, hordeolum).

In an advantageous embodiment, the inflammatory disease is an inflammatory disease or the skin or mucous membranes, particularly chosen in the group comprising or consisting of:
infections of the skin or skin appendages, in particular selected from the group comprising or consisting of folliculitis, furuncle, abscess, impetigo or whitlow;
inflammatory dermatoses, in particular selected from the group comprising or consisting of eczema, contact dermatitis, atopic dermatitis, rosacea or psoriasis;
inflammatory conditions of the eye and of the adnexa thereof, in particular selected from the group comprising or consisting of conjunctivitis, episcleritis, scleritis, keratitis, uveitis, blepharitis and hordeolum.

In a preferred embodiment, the inflammatory disease according to the invention is selected from the group comprising or consisting of rosacea, psoriasis, eczema, contact dermatitis and impetigo.

In one particular embodiment of the invention, the inflammatory disease is due to or associated with an infection. In one embodiment, the inflammatory disease is an inflammatory disease with an infectious component.

In one embodiment, the inflammatory disease is due to or associated with at least one Gram+ or Gram- type bacterium. In a preferred embodiment of the invention, the infectious component of the inflammatory disease is due to or associated with at least one bacterium chosen among the Gram+ bacteria belonging to the Escherichia, Pseudomonas, Klebsiella, Salmonella, Yersinia, or Vibrio genus; and the Gram- bacteria belonging to the Staphylococcus, Listeria, Serratia or Kocuria genus.

In a preferred embodiment, the infectious component of the inflammatory disease is due to or associated with a bacterium belonging to the Staphylococcus genus, preferentially Staphylococcus aureus.

In one embodiment, the inflammatory disease is due to or associated with at least one fungus. In a preferred embodiment of the invention, the infectious component of the inflammatory disease is due to or associated with a fungus belonging to the Candida or Pityriasis genus.

The invention also relates to a pharmaceutical composition comprising at least one peptide as described above, and one or a plurality of pharmaceutically acceptable excipients, for use in the prevention and/or treatment of an inflammatory disease.

The invention also relates to a medicament comprising at least one peptide as described above for use in the prevention and/or treatment of an inflammatory disease.

Preferably, the composition, pharmaceutical composition or medicament according to the present invention comprise a therapeutically effective quantity of a peptide according to the invention.

In one embodiment, the peptide, composition, pharmaceutical composition or medicament according to the present invention is used in combination with at least one further therapeutic agent to treat an inflammatory disease.

Examples of further therapeutic agents for treating an inflammatory disease include, but are not limited to, nonsteroidal anti-inflammatory drugs (NSAIDs) such as aspirin, ibuprofen, diclofenac, naproxen and ketorolac, celecoxib; steroidal anti-inflammatory drugs such as prednisone, dexamethasone, betamethasone, hydrocortisone aceponate, clobetasol propionate; and monoclonal anti-inflammatories such as anti-TNF agents (for example adalimumab, golumumab).

In one particular embodiment, the invention relates to a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a medicament or a pharmaceutical composition comprising such a peptide, for use in the treatment of burns and wounds, advantageously infected burns and wounds.

In one embodiment, the invention relates to a peptide having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, a medicament or a pharmaceutical composition comprising such a peptide, for use in the treatment of burns and wounds, advantageously infected burns and wounds.

In one embodiment, the invention relates to a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, a medicament or a pharmaceutical composition comprising such a peptide, for use in the treatment of burns and wounds, advantageously infected burns and wounds.

In one embodiment, the invention relates to a peptide with an anti-inflammatory activity and/or antimicrobial activity having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, a medicament or a pharmaceutical composition comprising such a peptide, for use in the treatment of burns and wounds, advantageously infected burns and wounds.

In one embodiment, the invention relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-Xs is an M or A residue;
-X₉ represents no residue or is an E residue;
-X₁₀ represents no residue or is a Q residue; and
functional derivatives and pharmaceutically acceptable salts of said peptide, a medicament or a pharmaceutical composition comprising such a peptide, for use in the treatment of burns and wounds, advantageously infected burns and wounds.

According to a particular form of the invention, the peptide as described above is suitable for use for the prevention or treatment of a disease, wherein the prevention or treatment consists of simultaneously obtaining the following effects:
i) an antimicrobial action, for example bacteriostatic, bactericidal, fungistatic or fungicidal; and
ii) a reduction in immune cell recruitment on an inflammation site.

The peptides according to the invention, when with an antimicrobial and anti-inflammatory activity, offer the advantage of being suitable for being administered to a patient suffering from an inflammatory disease due to or associated with an infection, without being liable to mask the signs of onset or worsening of an infection, unlike steroidal or nonsteroidal type anti-inflammatory drugs.

In one embodiment, the invention relates to a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for the prevention and/or treatment of a microbial infection, particularly an infection caused by a micro-organism such as a bacterium or a fungus.

In one embodiment, the invention relates to a peptide having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, for the prevention and/or treatment of a microbial infection, particularly an infection caused by a microorganism such as a bacterium or a fungus.

In one embodiment, the invention relates to a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue
-X₆ is an A, K or D residue;
-X₇ is a V or P residue;
functional derivatives and pharmaceutically acceptable salts of said peptide, for the prevention and/or treatment of a microbial infection, particularly an infection caused by a microorganism such as a bacterium or a fungus.

In one embodiment, the invention relates to a peptide having a sequence of 26 to 100 amino acids comprising the sequence A-L-X₁-X₂-T-L-X₃-K-K-V-G-X₄-X₅-X₆-G-K-A-X₇-L-N-A-V-T-N-Xs-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, for the prevention and/or treatment of a microbial infection, particularly an infection caused by a microorganism such as a bacterium or a fungus.

In one embodiment, the invention relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for the prevention and/or treatment of a microbial infection, particularly an infection caused by a microorganism such as a bacterium or a fungus.

In a preferred embodiment of the invention, the residue X₁ is W in SEQ ID NOs 1 and 29 to 32, the residue X₂ is K in SEQ ID NOs 1 and 29 to 32, the residue X₃ is L in SEQ ID NOs 1 and 29 to 32, the residue X₄ is K or A in SEQ ID NOs 1 and 29 to 32, the residue X₅ is V in SEQ ID NOs 1 and 29 to 32, the residue X₆ is A in SEQ ID NOs 1 and 29 to 32, and/or the residue X₇ is V in SEQ ID NOs 1, 29, 31 and 32. In a preferred embodiment of the invention, the residue X₉ represents the E residue and the residue X₁₀ represents the Q residue in SEQ ID NO 1.

In a further preferred embodiment of the invention, the residue X₉ represents no amino acid in SEQ ID NO 1. In a preferred embodiment of the invention, the residue X₁₀ represents no amino acid in SEQ ID NO 1. In a preferred embodiment of the invention, the residue X₉ is E and the residue X₁₀ is absent in SEQ ID NO 1. In a preferred embodiment of the invention, the residue X₉ represents the E residue and the residue X₁₀ represents no amino acid in SEQ ID NO 1.

In a further preferred embodiment of the invention, the residues X₉ and X₁₀ represent no amino acid.

In one embodiment, the peptide for the prevention and/or treatment of a microbial infection is a peptide comprising a sequence selected from the group comprising or consisting of the sequences SEQ ID NOs: 2 to 8.

In one particular embodiment of the invention, the peptide as described above is suitable for use for the treatment of a bacterial infection due to a bacterium belonging to the genus: Escherichia, Pseudomonas, Klebsiella, Salmonella, Yersinia, Vibrio, Staphylococcus, Bacillus, Listeria, Enterococcus, Kocuria, Serratia, Enterobacter, or Micrococcus.

In an even more particular embodiment of the invention, the peptide as described above is suitable for use for the treatment of an infection at least caused by a bacterium selected from the group comprising but not limited to, Staphylococcus aureus, Escherichia coli, Klebsiella oxytoca, Klebsiella pneumoniae Yersinia ruckeri, Staphylococcus epidermidis, Kocuria rhizophila, Bacillus subtilis, Clostridium difficile, Acinetobacter baumanii, Pneumococci, Serratia marcescens, Enterobacter aerogenes, Enterobacter cloacae, and Yersenia pestis.

The peptide as described above is also suitable for use for the treatment of a fungal infection caused for example by a fungus belonging to the Candida, Tricophyton, Microsporum, Epidermophyton, or Aspergillus genus.

In one particular embodiment of the invention, the peptide as described above is suitable for use for the treatment of an infection at least caused by a fungus selected from the groups comprising but not limited to Candida albicans, Candida glabrata or Pityriasis versicolor.

The invention further relates to a medicament or a pharmaceutical composition comprising a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for the prevention and/or treatment of a microbial infection, particularly an infection caused by a microorganism such as a bacterium or a fungus.

In one embodiment, the invention relates to a medicament or a pharmaceutical composition comprising a peptide having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue
-X₆ is an A, K or D residue;
functional derivatives and pharmaceutically acceptable salts of said peptide, for use in the prevention and/or treatment of a microbial infection, particularly an infection due to a microorganism such as a bacterium or a fungus.

In one embodiment, the invention relates to a medicament or a pharmaceutical composition comprising a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, a medicament or a pharmaceutical composition comprising such a peptide, for use in the prevention and/or treatment of a microbial infection, particularly an infection due to a microorganism such as a bacterium or a fungus.

In one embodiment, the invention relates to a medicament or a pharmaceutical composition comprising a peptide having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, a medicament or a pharmaceutical composition comprising such a peptide, for use in the prevention and/or treatment of a microbial infection, particularly an infection due to a microorganism such as a bacterium or a fungus.

In one embodiment, the invention relates to a medicament or a pharmaceutical composition comprising a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, for use in the prevention and/or treatment of a microbial infection, particularly an infection due to a microorganism such as a bacterium or a fungus.

Furthermore, the invention relates to a peptide with an angiogenic activity having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A (SEQ ID NO: 30), X₇-L-N-A-V-T-N-X₈-A (SEQ ID NO: 31), X₇-L-N-A-V-T-N-X₈-A-N-Q-N-X₉-X₁₀ (SEQ ID NO: 32), or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a pharmaceutical composition or a medicament comprising at least one such peptide, for the prevention and/or treatment of an angiogenesis-related disease.

In one embodiment, the invention relates to a peptide having a sequence of 17 to 100 amino acids comprising the sequence A-L-X₁-X₂-T-L-X₃-K-K-V-G-X₄-X₅-X₆-G-K-A (SEQ ID NO: 33), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, a pharmaceutical composition or a medicament comprising at least one such peptide, for the prevention and/or treatment of an angiogenesis-related disease.

In one embodiment, the invention relates to a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 34), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, a pharmaceutical composition or a medicament comprising at least one such peptide, for the prevention and/or treatment of an angiogenesis-related disease.

In one embodiment, the invention relates to a peptide having a sequence of 26 to 100 amino acids comprising the sequence A-L-X₁-X₂-T-L-X₃-K-K-V-G-X₄-X₅-X₆-G-K-A-X₇-L-N-A-V-T-N-Xs-A (SEQ ID NO: 35), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, a pharmaceutical composition or a medicament comprising at least one such peptide, for the prevention and/or treatment of an angiogenesis-related disease.

In one embodiment, the invention relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, a pharmaceutical composition or a medicament comprising at least one such peptide, for the prevention and/or treatment of an angiogenesis-related disease.

The formation of new blood vessels contributes to numerous malignant, ischemic, inflammatory, infectious and immune disorders. In a preferred embodiment, the invention relates to the peptide as described above for use in the prevention or treatment of an angiogenesis-related disease, preferably a peptide having a sequence of 29 to 100 amino acids comprising SEQ ID NO: 1, preferably comprising a sequence selected from the group comprising or consisting of the sequences SEQ ID NOs: 2, 3, 4, 5, 6, 7 or 8. Angiogenesis is a complex process whereby new vessels are formed, typically from the pre-existing vascular system. Angiogenesis plays an essential role in certain physiological processes such as the wound healing process. Besides the wound healing processes, numerous conditions have been described as having a component or a stage associated with the angiogenesis phenomenon.

The term “angiogenesis-related disease” as used in this document includes cancers, retinopathy type ophthalmological disorders (particularly retinopathies and macular degeneration), atherosclerosis, arthrosis, rheumatoid arthritis, psoriasis, or delayed healing-related conditions.

In one particular embodiment, the invention relates to a peptide, a pharmaceutical composition or a medicament as described above for the treatment of cancers, particularly for inhibiting tumour growth and/or metastasis formation in cancer patients. Indeed, it is established that angiogenesis is not merely essential for tumour growth but is also involved in progression from the benign stage to the invasive cancer stage. The possibility of inhibiting angiogenesis makes it possible to reduce tumour growth and metastatic cancer progression. Examples of cancers include, but are not limited to, cancers of the colon; lung; pancreas; breast; kidney such as renal cell carcinoma; skin such as basal cell carcinoma or melanoma; and glioblastoma. Advantageously, the peptide, pharmaceutical composition or medicament according to the invention are used to treat malignant tumours that induce new blood vessel formation.

The peptide, pharmaceutical composition or medicament according to the invention may be administered by injection, such as for example intravenous, intramuscular, subcutaneous injection, or directly into the tissue.

In one embodiment, the peptides according to the invention, regardless of the embodiment, are in a form acceptable for topical, systemic, oral, subcutaneous, intravitreal, intradermal, transdermal, intraperitoneal, intramuscular administration or administration by inhalation.

The pharmaceutical composition or medicament according to the invention may be in the form of tablets, capsules, hard capsules, granulates, suspensions, emulsions, solutions, gels, drops, pastes, ointments, creams, plasters, injectables, implants, patches.

The pharmaceutical composition or medicament according to the invention may comprise one or a plurality of pharmaceutically acceptable routine excipients.

In one embodiment, the composition, pharmaceutical composition or medicament according to the present invention comprises one or more pharmaceutically acceptable vehicles for a formulation suitable for topical administration. The pharmaceutically acceptable excipients may particularly be any excipient among those known to the those skilled in the art with a view to obtaining a composition for topical application in the form of a milk, cream, balm, oil, lotion, gel, foaming gel, ointment, or spray. In one particular embodiment, the excipients are chosen from the group consisting of an oil, fat, emulsion, gel, ointment, excipient based on nanoparticles or liposomes. The oils may particularly be chosen among silicone oils, mineral oils (paraffin, petrolatum, etc.) or vegetable oils and fats.

The pharmaceutical composition or medicament according to the invention may be prepared in the form of a water-in-oil (W/O) or oil-in-water (O/W) emulsion, a multiple emulsion such as for example, a water-in-oil-in-water (W/O/W) emulsion or an oil-in-water-in oil (O/W/O) emulsion, a micro-emulsion or in the form of a hydrodispersion or lipodispersion, gel or aerosol.

In a further embodiment, the composition, pharmaceutical composition or medicament according to the invention comprises one or more pharmaceutically acceptable vehicles for a formulation suitable for oral administration. Examples of forms suitable for oral administration include, but are not limited to, tablets (of which sustained-release tablets), hard capsules, powders, granules, pills (of which sugar-coated pills), capsules (of which soft gel capsules), oral suspensions, oral solutions, and other similar forms.

In one embodiment, the composition, the pharmaceutical composition or the medicament according to the present invention comprises one or more pharmaceutically acceptable vehicles for a formulation suitable for being injected. Examples of forms suitable for administration by injection include, but are not limited to, sterile aqueous solutions, dispersions, emulsions, suspensions, solid forms suitable for the preparation of solutions or suspensions by adding a liquid before use such as, for example, powders.

The pharmaceutical composition of the medicament according to the invention may also contain additives and formulation aids, such as emulsifiers, thickeners, gelling agents, water fixatives, spreading agents, stabilisers, colorants, fragrances and preservatives.

The doses are adjusted according to the effect sought and the condition targeted.

In one embodiment, the therapeutically effective quantity ranges from about 1 to 10000 mg/mL of peptide, composition, pharmaceutical composition or medicament according to the invention, preferably from about 5 to about 5000 mg/mL, preferably from about 10 to about 2000 mg/mL, preferably from about 20 to about 100 mg/mL of peptide, composition, pharmaceutical composition or medicament according to the invention.

In one embodiment, the therapeutically effective quantity ranges from about 1 to 10000 mg/g of peptide, composition, pharmaceutical composition or medicament according to the invention, preferably from about 5 to about 5000 mg/g, preferably from about 10 to about 2000 mg/g, preferably from about 20 to about 100 mg/g of peptide, composition, pharmaceutical composition or medicament according to the invention.

In one embodiment, the therapeutically effective quantity ranges from about 0.001 to 500 µM of peptide, composition, pharmaceutical composition or medicament according to the invention, preferably from about 0.01 to about 250 µM, preferably from about 0.1 to about 200 µM, preferably from about 0.5 to about 150 µM, composition, pharmaceutical composition or medicament according to the invention.

In one embodiment, the therapeutically effective quantity ranges from about 1 to 1000 µMof peptide, composition, pharmaceutical composition or medicament according to the invention, preferably from about 5 to about 500 µM, preferably from about 10 to about 250 µM, preferably from about 10 to about 150 µM, composition, pharmaceutical composition or medicament according to the invention.

In one embodiment, the therapeutically effective quantity ranges from about 1 to 100 µM of peptide, composition, pharmaceutical composition or medicament according to the invention, preferably from about 5 to about 75 µM, preferably from about 10 to about 50 µM.

In one embodiment, the therapeutically effective quantity ranges from about 10 to 1000 µMof peptide, composition, pharmaceutical composition or medicament according to the invention, preferably from about 50 to about 750 µM, preferably from about 100 to about 500 µM.

It is understood that the total daily use of peptide, composition, pharmaceutical composition or medicament according to the invention will be adjusted by the attending physician as part of their medical advice. The therapeutically effective dose specific to each patient will be dependent on a variety of factors including the disorder treated and the severity thereof; the activity of the compound used; the specific composition used; the age, weight, general state of health, sex and diet of the patient, the duration and method of administration; the duration of the treatment; the medicaments used in combination or coinciding with the compound used, and further similar factors known in the medical field. For example, it is common in this field to start with compound doses lower than the recommended doses to achieve the therapeutic effect sought and gradually increase the dosage until the effect has been achieved. However, the daily dosage of the compounds may vary over a vast range from about 1 to about 10000 mg per adult per day, preferably from about 5 to about 5000, preferably from about 10 to about 2000 mg, more preferentially from about 20 to about 100 mg per adult per day. Preferably, the composition comprises 1, 10, 20, 50, 100, 250, 500, 1000 and 2000 mg of the active ingredient for the symptomatic adjustment of the dosage to be administered to the patient to be treated. A medicament contains typically from about 1 to about 10000 mg of active ingredient, preferably from 5 to 5000, preferably from 10 to 2000 mg of active ingredient. An effective quantity of the medicament is ordinarily supplied at a dose ranging from about 0.01 mg/kg to about 100 mg/kg of body weight per day, preferably from about 0.05 mg/kg to about 40 mg/kg, preferably from about 0.1 mg/kg to 20 mg/kg of body weight per day, more preferentially from about 0.2 to about 1 mg/kg of body weight per day.

In one embodiment, the daily dose of the peptide according to the invention, the composition, pharmaceutical composition or medicament according to the present invention is adjusted according to the potential disorders at the level of the organs and systems, such as the kidney, liver and/or blood disorders of the patient.

In one embodiment, the peptide, the composition, pharmaceutical composition or medicament according to the present invention is administered to the subject at least once per day. For example, the peptide, composition, pharmaceutical composition or medicament according to the invention may be administered once per day, two times or three times per day. Preferably, the peptide, composition, pharmaceutical composition or medicament according to the invention is administered once per day. In a further embodiment, the peptide, composition, pharmaceutical composition or medicament according to the present invention is administered to the subject at least once per week. For example, the peptide, composition, pharmaceutical composition or medicament according to the invention may be administered once per week, two times, three times, four times or up to seven times per week.

The invention also relates to a method for the prevention and/or treatment of an inflammatory disease in a subject in need thereof, comprising the administration of an effective therapeutic dose of at least one peptide, medicament or pharmaceutical composition according to the invention.

In one embodiment, the method according to the invention for treating an inflammatory disease comprises the administration to the subject of a therapeutically effective quantity of a peptide according the invention as described above, preferably a peptide having a sequence of 29 to 100 amino acids comprising the sequence SEQ ID NO: 2, 3, 4, 5, 6, 7 or 8.

In one embodiment, the peptide, the composition, the pharmaceutical composition or the medicament according to the invention is administered to the subject.

In one embodiment, the peptide, the medicament or the pharmaceutical composition according to the invention is administered to a subject suffering or liable to suffer from an inflammatory disease. In one embodiment, the subject is an at-risk subject for the onset of an inflammatory disease.

In one embodiment, the subject has not yet been treated with another treatment for the inflammatory disease according to the invention. In a further embodiment, the subject has already been treated with another treatment for the inflammatory disease according to the invention.

The invention further relates to a method for the prevention and/or treatment of a microbial infection comprising the administration of an effective therapeutic dose of at least one peptide, medicament or pharmaceutical composition according to the invention.

In one embodiment, the method according to the invention for treating a microbial infection comprises the administration to the subject of a therapeutically effective quantity of a peptide according the invention as described above, preferably a peptide having a sequence of 29 to 100 amino acids comprising the sequence SEQ ID NO: 2, 3, 4, 5, 6, 7 or 8.

In one embodiment, the microbial infection is a bacterial or fungal infection as described above.

In one embodiment, the peptide, the medicament or pharmaceutical composition according to the invention is administered to a subject suffering or liable to suffer from a microbial infection. In one embodiment, the subject is an at-risk subject for the onset of a microbial infection.

In one embodiment, the subject has not yet been treated with another treatment for the microbial infection according to the invention. In a further embodiment, the subject has already been treated with another treatment for the microbial infection according to the invention.

The invention further relates to a method for the prevention and/or treatment of an angiogenesis-related disease comprising the administration of an effective therapeutic dose of at least one peptide, medicament or pharmaceutical composition according to the invention.

In one embodiment, the method according to the invention for treating an angiogenesis-related disease comprises the administration to the subject of a therapeutically effective quantity of a peptide according the invention as described above, preferably a peptide having a sequence of 29 to 100 amino acids comprising the sequence SEQ ID NO: 2, 3, 4, 5, 6, 7 or 8.

In one embodiment, the peptide, the medicament or the pharmaceutical composition according to the invention is administered to a subject suffering or liable to suffer from an angiogenesis-related disease. In one embodiment, the subject is an at-risk subject for the onset of an angiogenesis-related disease.

In one embodiment, the subject has not yet been treated with another treatment for the angiogenesis-related disease according to the invention. In a further embodiment, the subject has already been treated with another treatment for the angiogenesis-related disease according to the invention.

The invention further relates to the use of a peptide having a sequence of 17 to 100 amino acids comprising the sequence A-L-X₁-X₂-T-L-X₃-K-K-V-G-X₄-X₅-X₆-G-K-A-φ (SEQ ID NO: 29), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-φ is X₇-L-N-A,X₇-L-N-A-V-T-N-X₈-A, X₇-L-N-A-V-T-N-X₈-A-N-Q-N-X₉-X₁₀, or is absent;
and wherein:
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, as an antifungal or antibacterial agent.

In one embodiment, the invention relates to a peptide having a sequence of 17 to 100 amino acids comprising the sequence A-L-X₁-X₂-T-L-X₃-K-K-V-G-X₄-X₅-X₆-G-K-A (SEQ ID NO: 30), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
functional derivatives and pharmaceutically acceptable salts of said peptide, wherein the peptide is not a peptide of sequence SEQ ID NO: 2, as an antifungal or antibacterial agent.

In one embodiment, the invention relates to a peptide having a sequence of 21 to 100 amino acids comprising the sequence A-L-X₁-X₂-T-L-X₃-K-K-V-G-X₄-X₅-X₆-G-K-A-X₇-L-N-A (SEQ ID NO: 31), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
functional derivatives and pharmaceutically acceptable salts of said peptide, wherein the peptide is not a peptide of sequence SEQ ID NO: 2, as an antifungal or antibacterial agent.

In one embodiment, the invention relates to a peptide having a sequence of 26 to 100 amino acids comprising the sequence A-L-X₁-X₂-T-L-X₃-K-K-V-G-X₄-X₅-X₆-G-K-A-X₇-L-N-A-V-T-N-X₈-A (SEQ ID NO: 32), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
functional derivatives and pharmaceutically acceptable salts of said peptide, wherein the peptide is not a peptide of sequence SEQ ID NO: 2, as an antifungal or antibacterial agent.

In one embodiment, the invention relates to a peptide having a sequence of 29 to 100 amino acids comprising the sequence A-L-X₁-X₂-T-L-X₃-K-K-V-G-X₄-X₅-X₆-G-K-A-X₇-L-N-A-V-T-N-X₈-A-N-Q-N-X₉-X₁₀ (SEQ ID NO: 1), wherein:
-X₁ is a W or A residue;
-X₂ is a K or D residue;
-X₃ is an L or A residue;
-X₄ is a K, A or P residue;
-X₅ is a V or A residue;
-X₆ is an A, K or D residue;
-X₇ is a V or P residue;
-X₈ is an M or A residue;
-X₉ represents no residue or is an E residue;
-X₁₀ represents no residue or is a Q residue; and
functional derivatives and pharmaceutically acceptable salts of said peptide, wherein the peptide is not a peptide of sequence SEQ ID NO: 2, as an antifungal or antibacterial agent.

In a preferred embodiment of the invention, the residue X₁ is W in SEQ ID NOs 1 and 29 to 32, the residue X₂ is K in SEQ ID NOs 1 and 29 to 32, the residue X₃ is L in SEQ ID NOs 1 and 29 to 32, the residue X₄ is K or A in SEQ ID NOs 1 and 29 to 32, the residue X₅ is a V residue in SEQ ID NOs 1 and 29 to 32, the residue X₆ is A in SEQ ID NOs 1 and 29 to 32, and/or the residue X₇ is V in SEQ ID NOs 1, 29, 31 and 32. In a preferred embodiment of the invention, the residue X₉ represents the E residue and the residue X₁₀ represents the Q residue in SEQ ID NO 1.

In a further preferred embodiment of the invention, the residue X₉ represents no amino acid in SEQ ID NO 1. In a preferred embodiment of the invention, the residue X₁₀ represents no amino acid in SEQ ID NO 1. In a preferred embodiment of the invention, the residue X₉ is E and the residue X₁₀ is absent in SEQ ID NO 1. In a preferred embodiment of the invention, the residue X₉ represents the E residue and the residue X₁₀ represents no amino acid in SEQ ID NO 1.

In a further preferred embodiment of the invention, the residues X₉ and X₁₀ represent no amino acid.

In one embodiment, the peptide as described above is suitable for use for the preparation of an antifungal or antibacterial formulation.

In one embodiment, the peptide as described above is particularly used for disinfecting surfaces (for example walls, doors, medical equipment), a liquid (for example water) or a gas (for example an anaesthetic gas).

The present invention also relates to a kit comprising a peptide according to the invention, a nucleic acid, a vector, a composition, a pharmaceutical composition or a medicament as described above.

According to one embodiment, the kit also comprises an apparatus for administering the peptide, nucleic acid, vector, composition, pharmaceutical composition or medicament to a subject.

According to one embodiment, the kit further comprises instructions for the administration of the peptide, nucleic acid, vector, composition, pharmaceutical composition or medicament to said subject.

In one embodiment, the kit comprises an additional therapeutic agent. According to one embodiment, the additional therapeutic agent is a further agent for the treatment of the inflammatory disease according to the invention. According to a further embodiment, the additional therapeutic agent is a further agent for the treatment of the microbial infection according to the invention.

### BRIEF DESCRIPTION OF THE DRAWINGS

FIG. 1 shows the survival of mouse and human neutrophils (A), T lymphocytes (B), monocytes (C) and NK cells (D), in the presence of increasing concentrations of DA2N and DA2NEQ, as a percentage with respect to the number of live cells in the absence of peptide.

FIG. 2 shows the survival of mouse and human non-immune cells (epithelial cells), in the presence of increasing concentrations of DA2N and DA2NEQ peptide, as a percentage with respect to the number of live cells in the absence of peptide.

FIG. 3 shows the haemolytic activity of DA2N and DA2NEQ at 10 µMand 50 µM, on human red blood cells, as a percentage with respect to the positive control consisting of Triton X100 (100%).

FIG. 4 shows the number of immune cells in the peritoneal cavity 36 hours after 3 regular intraperitoneal (i.p.) injections of DA2N (dark grey) or DA2NEQ (light grey) peptide in a mouse model of peritonitis induced by i.p. injection of thioglycolate 3%. The negative control with respect to the peptide consists of injecting 0.15 M NaCl instead of the solution containing the peptide (black). The peritonitis induction control consists of mice non-induced for peritonitis (white); i.p. injection of NaCl.

FIG. 5 shows the protocol used to test the efficacy of the peptides in a contact eczema model.

FIG. 6 shows the relative variation in ear thickness in a mouse contact eczema model induced with DNCB 1%, after administering DA2N (A) or DA2NEQ (B). The contact eczema induction control corresponds to mice not treated with DNCB1% (line with triangle symbol) and the negative control corresponds to mice treated with peptide-free DNCB1% (line with square symbol).

FIG. 7 shows, in a mouse contact eczema model, the effect of administering DA2N on the number of immune cells (A) at the level of the ear, (B) in the systemic circulation and (C) in the spinal cord, after inducing contact eczema by applying DNCB1% (light grey). The contact eczema induction control corresponds to mice not treated with DNCB1% (white) and the negative control corresponds to mice treated with peptide-free DNCB1% (dark grey).

FIG. 8 shows representative images of the effect of a topical application of DA2N and DA2NEQ peptides in Balb/c mice on psoriatic skin lesions. The images of the left-hand column represent the results on psoriatic skin (skin treated with Imiquimod for 6 days): positive control with no peptide application (A), application of DA2N peptide (B) and application of DA2NEQ peptide (C). The images of the right-hand column represent the results on healthy skin (with no Imiquimod treatment): negative control with no peptide application (A), application of DA2N peptide (B) and application of DA2NEQ peptide (C).

FIG. 9 represents an enlargement at the level of the treated regions representative of the skin lesions shown in FIG. 8.

FIG. 10 shows representative images of the effect of a topical application of DA2N and DA2NEQ peptides on angiogenesis conventionally observed on balb/c mice on psoriatic lesions. The images of the left-hand column represent the results on psoriatic skin (skin treated with Imiquimod for 6 days): positive control with no peptide application (A), application of DA2N peptide (B) and application of DA2NEQ peptide (C). The images of the right-hand column represent the results on healthy skin (with no Imiquimod treatment): negative control with no peptide application (A), application of DA2N peptide (B) and application of DA2NEQ peptide (C).

FIG. 11 shows representative images of aortic rings and of neo-vessel formation after 3 days of treatment with PBS (A), DA2N (B), DA2NEQ (C).

FIG. 12 shows neo-vessel formation inhibition over time on mouse aortas after adding DA2N and DA2NEQ at the concentration of 12.5 µM, as a percentage with respect to the initial diameter of the aortic rings.

FIG. 13 shows the survival of mouse bone marrow cells after 2 hours of incubation in the presence of DA2N, DA2NE, N(D), DA2Na, K12A peptides at a concentration of 12.5 µMor 25 µM, as a percentage with respect to the number of initial live cells. The positive and negative controls consist, respectively, of the incubation of the cells in the presence of DMSO 30% or PBS.

FIG. 14 shows the survival of mouse non-immune cells (epithelial cells) in the presence of increasing concentrations of DA2N, K12A and DA2Na. DA2N, DA2Na and K12A induce the death of 50% of mouse immune cells originating from bone marrow (LC₅₀) at a concentration of 5 µM, 3.75 µM and 6.5 µM, respectively.

FIG. 15 shows the haemolytic activity of DA2Na and K12A peptides at 10 µM and 50 µM on mouse red blood cells, as a percentage with respect to the positive control consisting of Triton X100 (100%).

### EXAMPLES

### Peptide Synthesis

The peptides used in the examples were synthesised with the FMOC/tBu strategy on solid substrate by an Activo P11 automatic synthesiser (Activotec). Purification is carried out by HPLC (Waters, C18 column) and peptide identity is verified by MALDI-TOF spectrometry (MS Voyager Applied Biosystems). Due to the presence of a tryptophan residue in the sequence, the concentrations of the solutions are determined by UV spectroscopy (Nanodrop, Labtech. Com).

### Example 1: In Vitro Study of the Immunosuppressant Activity of DA2N and DA2NEQ

The cytotoxic effects of DA2N (SEQ ID NO: 4) and DA2NEQ (SEQ ID NO: 2) were evaluated on mouse and human immune cells. In order to demonstrate that the cytotoxic effects are specific to immune cells, the cytotoxic effects were also evaluated on non-immune cells.

### 1.1 Materials and Methods

The mouse immune cells are obtained from C57BL6 mouse bone marrow. The human immune cells were purified from the whole blood of healthy donors (blood bank, IMSS, Cuernavaca, Mexico).

The following non-immune cells were extracted after sacrificing C57BL6 mice or in a surgical context: epithelial cells, adipocytes, hepatocytes.

The peptides are incubated at 37° C., 5% CO2 in the presence of 500,000 immune and non-immune cells in RPMI medium at varied concentrations and at varied times. Peptide-free cells are used as the negative control and cells incubated for 5 min at 50° C. or in the presence of DMSO (30%) are used as the positive control. After incubation, the cells are washed then labelled with propidium iodide so as to measure the cell viability. The cell viability is analysed by counting or with flow cytometry in the presence of antibodies coupled with fluorochromes corresponding to characteristic markers of each cell population. The LC₅₀ is then calculated as being the concentration of DA2N and DA2NEQ peptides inducing the death of 50% of each one of the immune cell populations.

Cytometry data processing is carried out with FlowJo software.

### 1.2 Results

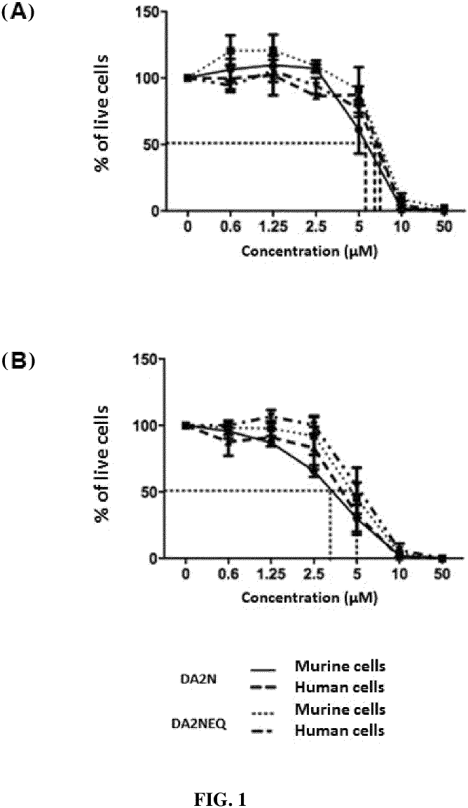

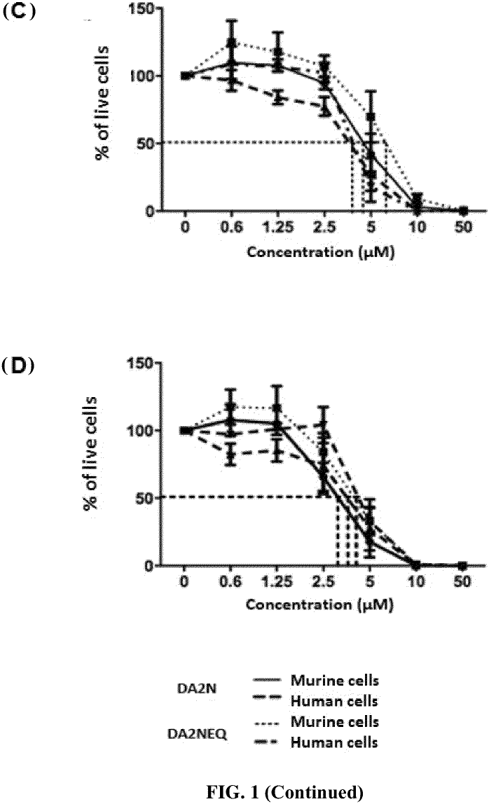

The survival of mouse and human immune cells in the presence of increasing concentrations of DA2N and DA2NEQ is shown in FIG. 1.

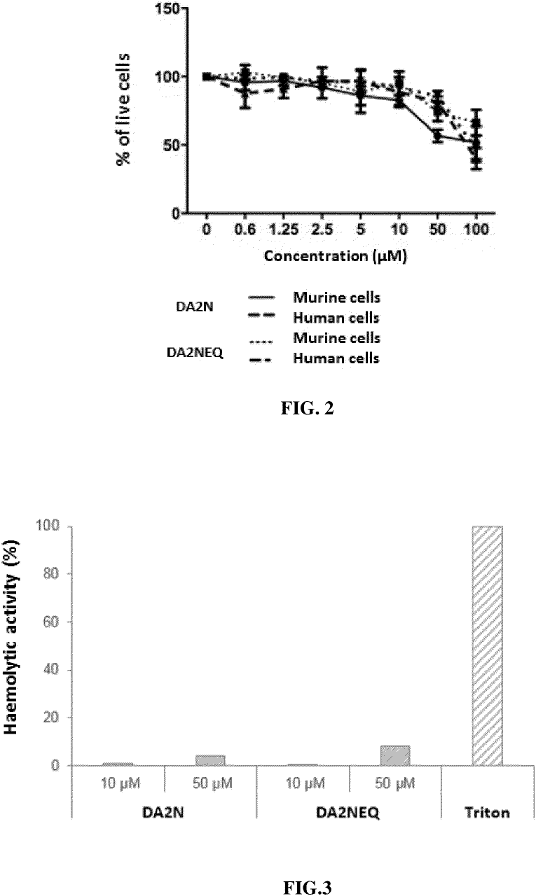

The survival of mouse and human non-immune cells in the presence of increasing concentrations of DA2N and DA2NEQ is shown in FIG. 2.

By comparing the results shown in FIG. 1 and in FIG. 2, the results obtained demonstrate that DA2N and DA2NEQ, at doses of the micromolar order, induce the death of immune cells but not of non-immune cells.

Table 1 shows the DA2N and DA2NEQ peptide concentrations inducing the death of 50% of mouse and human immune cells (LC₅₀).

TABLE 1

LC₅₀ on immune cells.
LC50 (µM)

| | DA2N | | DA2NEQ | |
|---|---|---|---|---|
| | Human cells | Mouse cells | Human cells | Mouse cells |
| Neutrophils | 7.5 µM | 6 µM | 7 µM | 7.5 µM |
| T lymphocytes | 4 µM | 3 µM | 5 µM | 5 µM |
| Monocytes | 4 µM | 5 µM | 4 µM | 7 µM |
| NK cells | 4 µM | 3 µM | 4 µM | 4 µM |

Thus, these results demonstrate that the peptides according to the invention are endowed with an immunosuppressant activity.

### Example 2: In Vitro Study of the Antimicrobial Activity of DA2N and DA2NEQ

### 2.1 Materials and Methods

### Measurement of Antibacterial Activity

The following strains were used for the antibacterial tests:

Escherichia coli (ATCC 8739, ATCC ML35p and P7 BLSE), Pseudomonas aeruginosa (ATCC 9027 and ATCC 27853), Klebsiella oxytoca (CIP 7932), Klebsiella pneumoniae (CIP 52.211), Salmonella enterica (CIP 8297), Yersinia ruckeri (ATCC 29473), Vibrio parahemolyticus (IFREMER 01 01/252), Staphylococcus aureus (ATCC 6538, ST 1065 and MRSA), Staphylococcus epidermidis (BM 3302), Listeria monocytogenes (SOR 100), Enterococcus faecalis (CIP A186, CIP 103015)), Kocuria rhizophila (ATCC 9341), Lactococcus garviae (ATCC 43921), Bacillus subtilis (CIP 52.65).

For each strain, a 5 ml pre-culture in LB medium is prepared by inoculating a colony of the bacterial strain of interest. The pre-culture is incubated at 37° C. under stirring overnight. The culture is subcultured (30 µL) in 5 mL. The culture concentration is evaluated by measuring the optical density at 620 nm. The concentration is adjusted by dilution so as to obtain a suspension with an OD of 0.05 at 620 nm.

### Measurement of Minimum Inhibitory Concentration (MIC)

The MIC is determined using a growth inhibition test in liquid medium. The MIC is defined as being the lowest peptide concentration capable of inhibiting the bacterial strain tested, after incubation for 18 h at 37° C. The test was carried out in a sterile microtitration plate containing 96 wells. A series of increasing concentrations of each peptide (0.2 to 100 µM) was prepared beforehand in sterile MilliQ water. In each well, 10 µL of peptide was incubated with 90 µL of bacterial suspension (10⁷ cfu/mL) in yeast extract-free LB medium. The microplate was incubated at 37° C. for 18 h. The bacterial growth was evaluated by measuring the OD at 620 nm using a plate reader.

The negative growth inhibition control was obtained by replacing the solution containing the peptide by sterile MilliQ water. The positive control suitable for completely inhibiting the growth of the bacterial strains was obtained by replacing the solution containing the peptide by 10 µL of formaldehyde.

The results are interpreted by calculating the percentage of bacterial growth in each well according to the formula:

%Growth = (OD well - OD of T₀)/(OD of T₁₀₀-OD of T₀)*100 where

OD of T₁₀₀ = OD of growth control (bacterial suspension free from sample under test, 100% growth),
OD of T₀ = OD of sterility control (culture medium + bacterium + 10 µL formaldehyde, 0% growth) and
OD well = OD of well for which it is sought to calculate the growth percentage (bacterial suspension + peptide under test).

### Measurement of Minimum Bactericidal Concentration (MBC)

The MBC is defined as being the minimum peptide concentration leaving no surviving bacteria of the inoculum after incubation at 37° C. for 24 hours. This test consists of smearing the contents of the well wherein the peptide has inhibited bacterial growth on a Petri dish (LB agar), followed by incubation for 24 h at 37° C. The inhibition is referred to as bacteriostatic when the peptide inhibits bacterial growth but does not lyse the bacteria and it is referred to as bacteriolytic when the peptide has completely lysed the bacteria and therefore prevents regrowth of the bacteria in the culture medium after subculturing.

### 2.2 Results

The antimicrobial activity of DA2N and DA2NEQ was evaluated on various Gram-positive and Gram-negative reference bacterial strains. The results are shown in Table 2:

TABLE 2

Antimicrobial activities of DA2N and DA2NEQ

| | MIC (µM) | | MBC (µM) | |
|---|---|---|---|---|
| | DA2N | DA2NEQ | DA2N | DA2NEQ |
| Gram-negative | | | | |
| Escherichia coli ATCC 8739 | 3.12 | 3.12 | 3.12 | 6.25 |
| Escherichia coli ML35p | 3.12 | 6.25 | 6.25 | 6.25 |
| Pseudomonas aeruginosa ATCC 9027 | 12.5 | 12.5 | 12.5 | 25 |
| Klebsiella oxytoca CIP 7932 | 3.12 | 12.5 | 6.25 | >100 |
| Klebsiella pneumoniae CIP 5211 | 3.12 | 3.12 | | |
| Gram-positive | | | | |
| Staphylococcus aureus ATCC 6538 | 6.25 | 12.5 | 6.25 | 12.5 |
| Staphylococcus aureus ST 1065 | 12.5 | >100 | 12.5 | >100 |
| Staphylococcus epidermidis BM 3302 | 1 | >100 | 12.5 | >100 |
| Kocuria rhizophila ATCC 9341 | 3.12 | 6.25 | 3.12 | 6.25 |
| Bacillus subtilis CIP 52.65 | 6.25 | 6.25 | 6.25 | 6.25 |

These results demonstrate that DA2N and DA2NEQ exhibit an antimicrobial activity on Gram-positive and Gram-negative bacteria. Note that the antimicrobial activity of DA2N is generally greater than that of DA2NEQ, particularly with respect to Escherichia coli P7 (ESBL). This result is of particular interest given that this strain is resistant to most β-lactamases.

### Example 3: In Vitro Study of the Haemolytic Activity of DA2N and DA2NEQ

### 3.1 Materials and Methods

### Measurement of Haemolytic Activity

The haemolytic activity of antimicrobial peptides was demonstrated using mouse or human red blood cells from healthy adult donors. Mouse or donor blood is washed three times in 10 volumes of PBS at 4° C. by centrifugation at 800 g for 10 minutes. The blood sediment is finally diluted to 4% (v/v) in PBS buffer. The test was carried out as follows: either 10 µM, or 50 µM of peptide was added to 100 µL of red blood cell suspension.

After 1h of incubation at 37° C. followed by centrifugation (800 g, 15 min), the absorbance of the supernatant was measured at 550 nm.

The negative control for this test (0% haemolysis) contains 5 µL of PBS buffer instead of the solution containing the peptide and the positive control (100% haemolysis) contains 5 µL of 1% Triton X100 instead of the solution containing the peptide.

Measurement of LC₅₀

The mouse red blood cells are obtained from C57BL6 mouse bone marrow.

The human red blood cells were purified from the whole blood of healthy donors (blood bank, IMSS, Cuernavaca, Mexico).

The peptides are incubated at 37° C., 5% CO₂ in the presence of 500,000 red blood cells in RPMI medium at varied concentrations and at varied times. Peptide-free cells are used as the negative control and cells incubated for 5 min at 50° C. or in the presence of DMSO (30%) are used as the positive control. The LC₅₀ is then calculated as being the concentration of DA2N and DA2NEQ peptides inducing the death of 50% of the red blood cells.

### 3.2 Results

FIG. 3 shows the percentage of haemolysis of human red blood cells by DA2N and DA2NEQ peptides, with respect to the positive control with reference to 100%. The results show that the DA2N and DA2NEQ peptides do not induce the haemolysis of human (FIG. 3) or mouse (results not shown) red blood cells.

Table 3 shows the DA2N and DA2NEQ peptide concentrations inducing the death of 50% of human and mouse red blood cells (LC₅₀).

TABLE 3

LC₅₀ on red blood cells
LC50 (µM)

| DA2N | | DA2NEQ | |
|---|---|---|---|
| Human red blood cells | Mouse red blood cells | Human red blood cells | Mouse red blood cells |
| 750 µM | 750 µM | 750 µM | 750 µM |

Thus, these results demonstrate that the peptides according to the invention have a negligible haemolytic activity.

### Example 4: In Vivo Study of the Anti-inflammatory Activity of DA2N and DA2NEQ

### 4.1 Materials and Methods

The anti-inflammatory activity of DA2N and DA2NEQ was evaluated in a mouse model of non-infectious peritonitis. Non-infectious peritonitis is induced by intraperitoneal injection of thioglycolate 3%. Six hours after inducing peritonitis, three intraperitoneal injections of peptide (90 µg/injection/mouse) were carried out at regular intervals (every 6 h). At 36 h, the mice are sacrificed in order to observe the immune cell recruitment in the peritoneal cavity. The cells obtained from washing the peritoneal cavity are washed and then labelled with propidium iodide in order to measure the cell viability and in the presence of antibodies coupled with fluorochromes corresponding to characteristic markers of each cell population. The evaluation of the anti-inflammatory potential is based on the measurement of the number of neutrophils, monocytes and macrophages recruited in the mouse peritoneal cavity. The negative control with respect to the peptide for this test consists of 50 µL of 0.15 M NaCl instead of the solution containing the peptide. The peritonitis induction control consists of mice non-induced for peritonitis. Data processing is carried out with FlowJo software.

### 4.2 Results

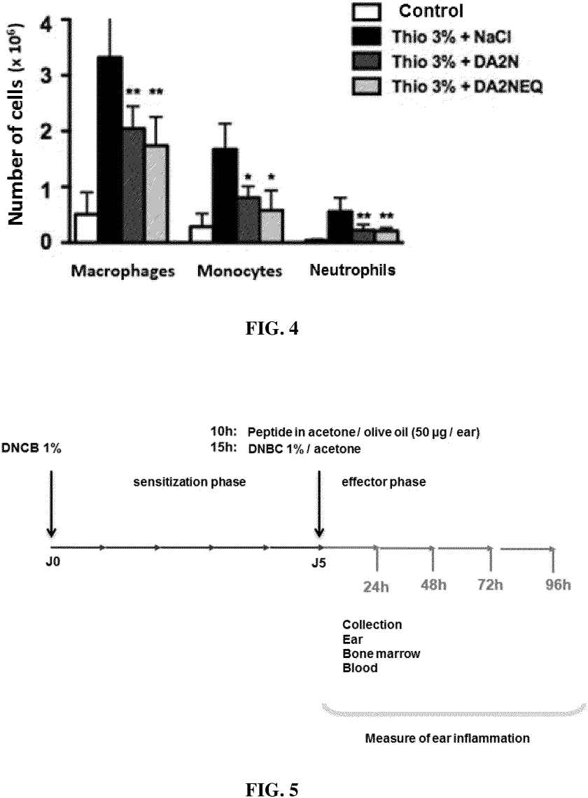

FIG. 4 shows the number of immune cells in the peritoneal cavity in the mouse model of thioglycolate-induced peritonitis, and therefore the effect of DA2N and DA2NEQ in an inflammatory disease model. The results demonstrate that, in vivo, the peptides induce a reduction in neutrophil and monocyte/macrophage recruitment in the peritoneal cavity after inducing peritonitis. The peptides according to the invention therefore have an anti-inflammatory activity.

In the light of the in vitro results, the inventors consider, without wishing to be bound to any theory, that the reduction in recruitment observed is due to the death of the immune cells under the effect of the DA2N and DA2NEQ peptides.

### Example 5: In Vivo Study of DA2N and DA2NEQ Activity in a Contact Eczema Model

### 5.1 Materials and Methods

The study is carried out to determine the activity of the peptides DA2N (SEQ ID NO: 4) and DA2NEQ (SEQ ID NO: 2) with respect to contact sensitisation. Mice are sensitised with DNCB 1% for 5 days. After this sensitization phase, 30 µL of a 1:1 olive oil/acetone mixture (vehicle) wherein 50 µg of DA2N or DA2NEQ peptide is dissolved is applied at the level of the ear. Five hours after applying the peptide, the contact reaction is induced by re-exposure to DNCB 1 % at the level of the ear. The intensity of the inflammatory reaction is evaluated locally 24, 48, 72 and 96 hours after re-exposure to DNCB by measuring ear thickness. Moreover, the number of immune cells (dendritic cells, T lymphocytes, neutrophils, monocytes, macrophages) is measured 24 h after re-exposure to DNCB in the ear at the level of the DNCB 1% application site (number of immune cells counted in both ears), in the blood (number of immune cells counted in 50 µL of blood) and in the bone marrow (number of immune cells counted in a tibia). The protocol is represented schematically in FIG. 5.

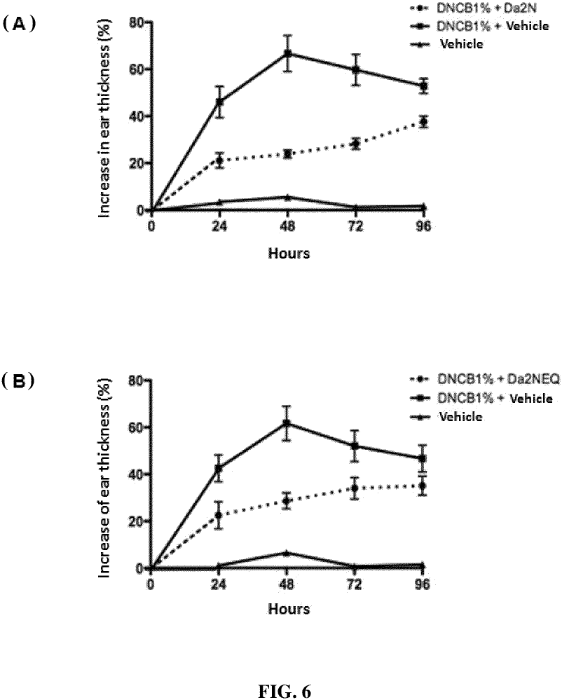

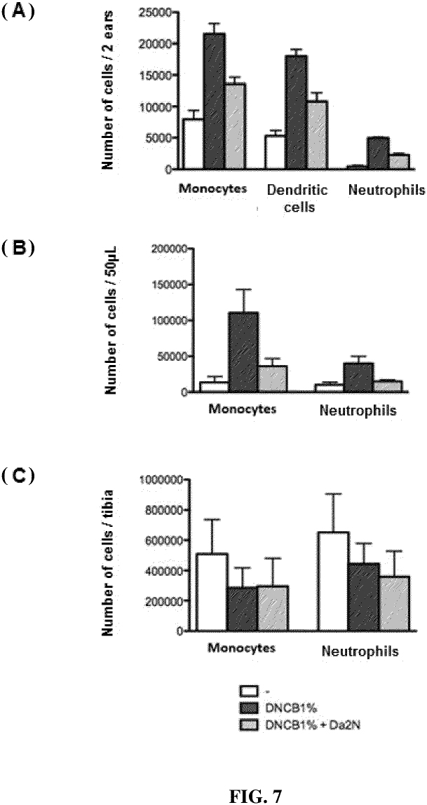

In order to count the number of cells, the ears are digested by adding collagenase IV and the number of immune cells is determined by flow cytometry using antibodies coupled with fluorochromes corresponding to characteristic markers of each cell population. The negative contact reaction inhibition control was obtained by replacing the solution containing the peptide with the 1:1 olive oil/acetone solution (indicated as “vehicle” in FIG. 6, and with the symbol “-” in FIG. 7).

### 5.2 Results

The results show a reduction in ear thickness (FIGS. 6A and B) after topical application of DA2N or DA2NEQ. Furthermore, FIG. 7 shows a reduction in the number of monocytes, dendritic cells and neutrophils, whether at the level of the ear (FIG. 7A), in the blood (FIG. 7B) or in the bone marrow (FIG. 7C) of the mice after topical application of DA2N.

The peptides according to the invention therefore have an anti-inflammatory effect in an in vivo inflammatory disease model.

### Example 6: In Vivo Study of DA2N and DA2NEQ Activity in a Psoriasis Model

### 6.1 Materials and Methods

The DA2N and DA2NEQ peptides were tested in an Imiquimod-induced psoriasis model described by Van der Fits (Van der Fits et al. 2009. Journal of immunology. 182(9):5836-5845) on female balb/c mice, aged 7 to 8 weeks.

31 mg of commercial cream containing 5% Imiquimod (Aldara, 3 M Pharmaceuticals) is applied topically on about 2.5 cm of the previously shaven ventral region of the mice for 6 days. The first psoriatic lesions appear on the 3rd day. The peptides resuspended in the cream are applied topically or intravenously (10 µg) for the first 3 days of Imiquimod treatment. Mice with no Imiquimod treatment and/or with no peptide application were used as controls. On the 6th day, the mice are sacrificed and the skin sampled is analysed by microscopy (skin lesions and angiogenesis).

### 6.2 Results

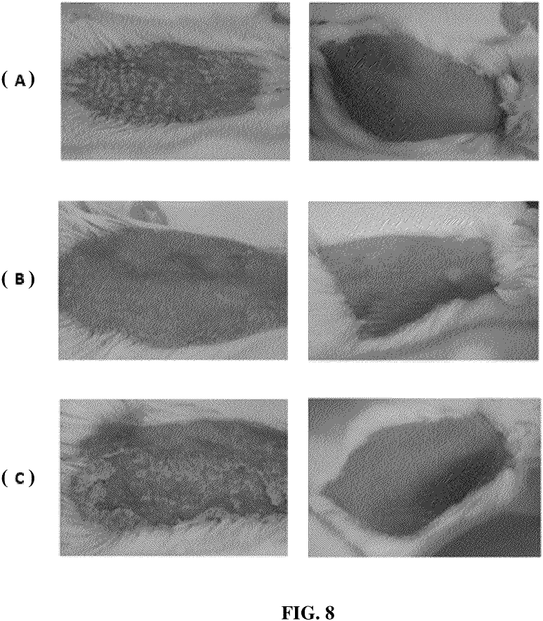

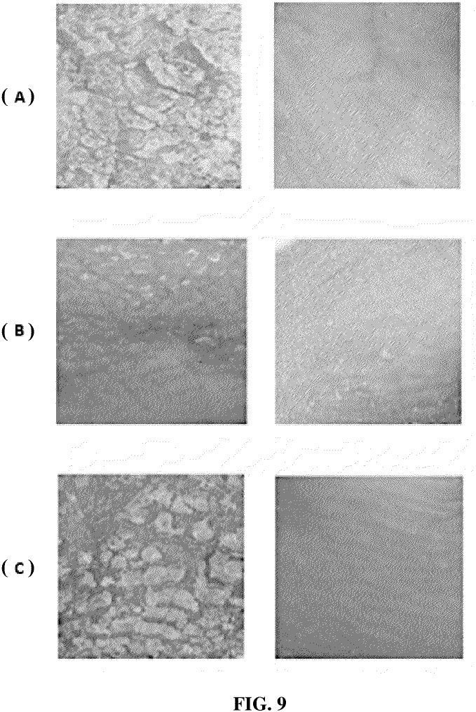

FIGS. 8 and 9 (enlargement) show representative images of a topical treatment with DA2N and DA2NEQ at a dose of 10 µg on psoriatic lesions induced by applying Imiquimod for 6 days. Compared to the positive control, DA2N reduces the psoriatic lesions conventionally observed after 6 days of Imiquimod treatment. DA2N and DA2NEQ have no effect on healthy skin.

### Example 7: Study of the Anti-angiogenic Activity of DA2N and DA2NEQ

### 7.1 Materials and Methods

The DA2N and DA2NEQ peptides were tested on the aortic ring model making it possible to study the influence of various molecules on new blood vessel formation. The aortas of C57BL/6 or Balb/c mice are sampled, washed with PBS then cut into rings. The rings are then deposited in a Matrigel matrix (BD® Matrigel™) in 24-well plates, incubated for 1h at 37° C., 5% CO₂, before adding 1 mL of DMEM (DMEM Advanced 1X, Gibco) supplemented with 5% foetal calf serum (FCS), 2 µM of glutamine, 100 U of penicillin and 50 pg/mL of streptomycin. The rings are then incubated at 37° C., 5% CO₂ for 3 days. The medium is then removed and replaced by medium containing or not the peptides at a concentration of 12.5 µM. The aortic rings are once again incubated for 3 days under the same conditions. Photos of the rings are taken each day to measure neo-vessel formation

Image processing and the measurement of the area formed by the neo-vessels are carried out using Image J 1.50i software.

### 7.2 Results

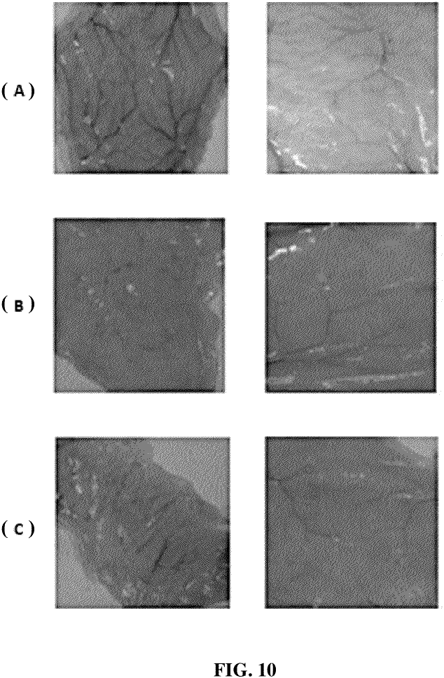

FIG. 10 shows representative images of a topical treatment with DA2N and DA2NEQ at a dose of 10 µg on angiogenesis, conventionally observed after applying Imiquimod for 6 days. Compared to the positive control, DA2N and DA2NEQ appear to reduce neo-vessel formation after 6 days of Imiquimod treatment. DA3N and DA2NEQ have no effect on the angiogenesis of healthy skin.

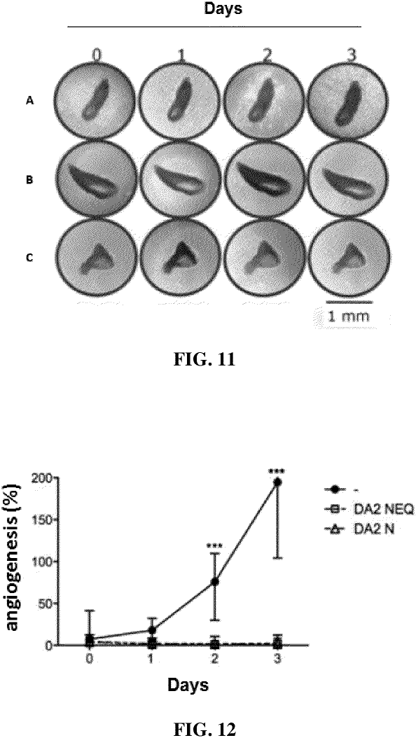

FIG. 11 shows representative images showing neo-vessel formation (angiogenesis) on D0, D1, D2 and D3 from aortic rings, after adding DA2N or DA2NEQ on D0. The control corresponds to neo-vessel formation, after adding only PBS. FIG. 12 shows the neo-vessel formation as a percentage with respect to the initial diameter of the aortic ring. Compared to the control (PBS), adding DA2N or DA2NEQ inhibits neo-vessel formation significantly.

### Example 8: In Vitro Study of the Immunosuppressant Activity of DA2NE, DA2N (D), K12A and DANa

The cytotoxic effects of DA2NE (SEQ ID NO: 3), DA2N (D) (SEQ ID NO: 19), K12A (SEQ ID NO: 11) and DANa (SEQ ID NO: 7) were evaluated on immune cells obtained from mouse bone marrow.

### 8.1 Materials and Methods

The mouse immune cells are obtained from C57BL/6 or Balb/c mouse bone marrow. The peptides are incubated at 37° C., 5% CO₂ in the presence of 500,000 immune cells in RPMI medium at varied concentrations for 2 hours. Peptide-free cells are used as the negative control and cells in the presence of DMSO (30%) are used as the positive control. After incubation, the cells are washed then labelled with Trypan blue so as to measure the cell viability by microscope counting. The LC₅₀ is then calculated as being the concentration of DA2N, K12A and DANa peptides inducing the death of 50% of the immune cells.

### 8.2 Results

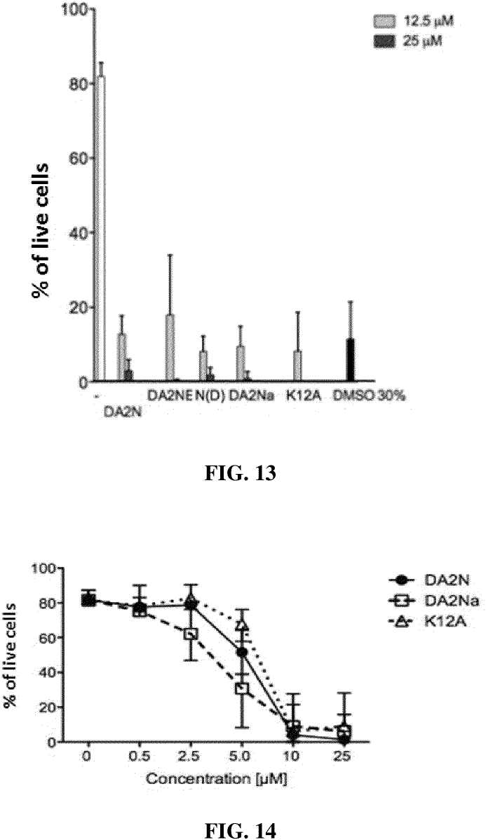

The survival of the immune cells obtained from mouse bone marrow in the presence of DA2NE, DA2N (D), K12A and DANa peptides at the concentrations of 12.5 and 25 µM is shown in FIG. 13 and compared to DA2N. The DA2NE, DA2N (D), K12A and DANa peptides appear to induce the death of immune cells at a concentration of 12.5 µM with a comparable efficacy to DA2N.

The survival of mouse non-immune cells in the presence of increasing concentrations of DA2N, K12A and DA2Na is shown in FIG. 14. DA2N, DA2Na and K12A induce the death of 50% of mouse immune cells originating from bone marrow (LC₅₀) at a concentration of 5 µM, 3.75 µM and 6.5 µM, respectively.

### Example 9: In Vitro Study of the Haemolytic Activity of DA2NE, DA2N (D), K12A and DANa

### 9.1 Materials and Methods

### Measurement of Haemolytic Activity

The haemolytic activity of the antimicrobial peptides was measured as per the protocol described in part 3.1 of Example 3.

### 9.2 Results

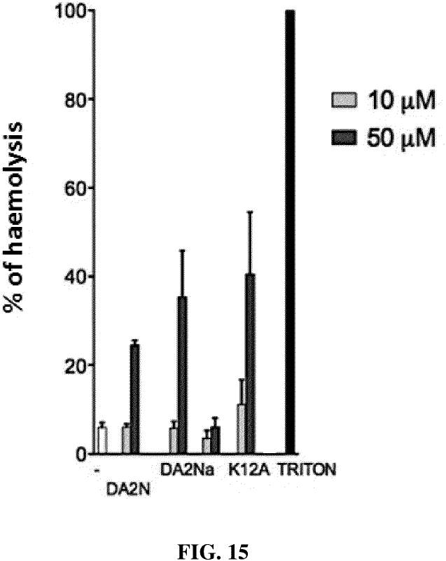

FIG. 15 shows the percentage of haemolysis of mouse red blood cells after incubation with the peptides for 1 hours, with respect to the positive control with reference to 100%. The results show that the DA2Na and K12A peptides do not exhibit significant haemolytic activity (less than 40% haemolysis) at the concentrations of 10 and 50 µM.

### Example 10: In Vitro Study of the Antimicrobial Activity of DA2NE, DA2Na, K4D, L7A, K12A, K12P, V13A, A14D, A14K, M25A, DA2NEQ (D) and DA2N (D)

The antimicrobial activity of DA2N (SEQ ID NO: 4), DA2NEQ (SEQ ID NO: 2), DA2NE (SEQ ID NO: 3), DA2Na (SEQ ID NO: 7), K4D (SEQ ID NO: 13), L7A (SEQ ID NO: 14), K12A (SEQ ID NO: 11), K12P (SEQ ID NO: 12), V13A (SEQ ID NO: 17), A14D (SEQ ID NO: 10), A14K (SEQ ID NO: 8), M25A (SEQ ID NO: 16), DA2NEQ (D) (SEQ ID NO: 18) and DA2N (D) (SEQ ID NO: 19) was evaluated on various microbial strains.

ScrL (NLGAKNQVAWEKKAATTKGNVNLKLLQMVAV; SEQ ID NO: 37) and ScrC (KGNVLVKAAMLTKVLAKNKGVQNANALTW; SEQ ID NO: 38) are random sequences of the original peptides used as negative controls.

Melittin (CAS number: 37231-28-0) and Gramicidin (CAS number: 1405-97-6) are used as positive controls.

### 10.1 Materials and Methods

### Preparation of Bacterial Suspensions

A 10 mL preculture in Muller-Hinton (MH) medium is prepared by inoculating a colony of the bacterial strain of interest. The preculture is incubated at 37° C. under stirring overnight. The culture is subcultured to 1:100 under the same conditions. The culture concentration is evaluated by measuring the turbidity at 600 nm (OD600 nm 1.0 = 5 × 10⁸ CFU/mL for E. coli (Gram-negative bacteria) and 1.5 × 10⁸ CFU/mL for S. aureus (Gram-positive bacteria). Once the exponential growth phase (OD600 nm = 0.3 - 0.7) has been achieved, the cultures are diluted in MH medium so as to obtain 10⁶ CFU/mL (final inoculum concentration).

### Measurement of MIC and MBC

The MIC and the MBC were determined as per the protocol described in part 2.1 of Example 2.

### 10.2 Results

The antimicrobial activity of DA2N and DA2NEQ is shown in Table 4:

TABLE 4

Antimicrobial activities of DA2N and DA2NEQ (ND: not determined)

| | DA2N | | DA2NEQ | | Melittin | |
|---|---|---|---|---|---|---|
| | MIC | MBC | MIC | MBC | MIC | MBC |
| Gram-negative | | | | | | |
| Escherichia coli ATCC 8739 | 0.8 | 0.8 | 1.6 | 1.6 | 2.5 | 2.5 |
| Escherichia coli K12 | 3.12 | 6.3 | 12.5 | 12.5 | 10 | 10 |
| Escherichia coli ML35p | 0.8 | 0.8 | 1.6 | 1.6 | 10 | 10 |
| Escherichia coli P7 (ESBL) | 1.6 | 12.5 | 12.5 | 50 | 6.25 | 6.25 |
| Pseudomonas aeruginosa ATCC 9027 | 3.12 | 3.12 | 6.3 | 6.3 | 5 | 5 |
| Pseudomonas aeruginosa ATCC 27853 | 12.5 | 50 | 50 | >100 | 50 | >50 |
| Klebsiella pneumoniae CIP 52.211 | 0.8 | 0.8 | 0.8 | 0.8 | 10 | 10 |
| Klebsiella oxytoca CIP 7932 | 3.12 | 6.25 | 12.5 | >100 | 25 | 50 |
| Salmonella enterica CIP 8297 | 12.5 | 25 | 50 | 100 | 50 | >50 |
| Yersinia ruckeri ATCC 29473 | 3.12 | 12.5 | 25 | >100 | 6.25 | 12.5 |
| Vibrio parahemolyticus IFREMER 01/252 | >50 | >50 | >100 | >100 | >50 | >50 |
| Gram-positive | | | | | | |
| Staphylococcus aureus ATCC 6538 | 1.6 | 3.12 | 6.3 | 12.5 | 5 | 5 |
| Staphylococcus aureus ST 1065 | 6.3 | 6.3 | 12.5 | 25 | 10 | 10 |
| Staphylococcus epidermidis BM 3302 | 6.3 | 25 | 12.5 | >50 | 10 | 10 |
| Staphylococcus aureus MRSA | 25 | >50 | 100 | >100 | 6.25 | 12.5 |
| Listeria monocytogenes SOR 100 | 25 | 25 | 100 | >100 | 1.5 | 3.12 |
| Enterococcus faecalis CIP A186 | 50 | >50 | 100 | >100 | 12.5 | 12.5 |
| Lactococcus garviae ATCC 43921 | 25 | 50 | 100 | >100 | 6.25 | 25 |
| Enterococcus faecalis CIP 103015 | 6.3 | 6.3 | 12.5 | 12.5 | 1.25 | 1.25 |
| Kocuria rhizophila ATCC 9341 | 0.8 | 1.6 | 1.6 | 1.6 | 0.8 | 0.8 |
| Bacillus subtilis CIP 52.65 | 6.25 | 6.25 | 25 | ND | ND | ND |

These results therefore confirm the results obtained in example 2 (Table 2) which demonstrate that DA2N and DA2NEQ have an antimicrobial activity on Gram-positive and Gram-negative bacteria.

The antimicrobial activity of DA2NE, DA2Na, K4D, L7A, K12A, K12P, V13A, A14D, A14K, M25A, DA2NEQ (D) and DA2N (D) is shown in Table 5:

TABLE 5

Antimicrobial activities of DA2NE, DA2Na, K4D, L7A, K12A, K12P, V13A, A14D, A14K, M25A, DA2NEQ (D) and DA2N (D)

| | Gram-negative | | | | Gram-positive | | | |
|---|---|---|---|---|---|---|---|---|
| | E. coli ATCC 8739 | | P. aeruginosa ATCC 9027 | | S. aureus ATCC 6538 | | E. faecalis CIP 103015 | |
| | MIC (µM) | MBC (µM) | MIC (µM) | MBC (µM) | MIC (µM) | MBC (µM) | MIC (µM) | MBC (µM) |
| DA2NE | 0.8 | 1.6 | 3.1 | 3.1 | 3.1 | 12.5 | 12.5 | 50 |
| DA2Na | 0.8 | 0.8 | 1.6 | 1.6 | 0.8 | 1.6 | 3.1 | 3.1 |
| K4D | 12.5 | 12.5 | 25 | 25 | 12.5 | 12.5 | 50 | 50 |
| L7A | 0.8 | 0.8 | 1.6 | 1.6 | 3.1 | 3.1 | 6.3 | 6.3 |
| K12A | 1.6 | 1.6 | 6.3 | 6.3 | 3.1 | 6.3 | 12.5 | 12.5 |
| K12P | 6.3 | 6.3 | 12.5 | 12.5 | 6.3 | 6.3 | >50 | >50 |
| V13A | 1.6 | 1.6 | 6.3 | 6.3 | 6.3 | 12.5 | 50 | 50 |
| A14D | 6.3 | 6.3 | 50 | >50 | >50 | >50 | >50 | >50 |
| A14K | 1.6 | 1.6 | 12.5 | 12.5 | 25 | 50 | >50 | >50 |
| M25A | 0.8 | 0.8 | 3.1 | 3.1 | 3.1 | 3.1 | 12.5 | 12.5 |
| Scr L | >50 | >50 | >50 | >50 | >50 | >50 | >50 | >50 |
| Scr C | >50 | >50 | >50 | >50 | >50 | >50 | >50 | >50 |
| DA2NEQ (D) | 0.8 | 0.8 | 1.6 | 3.1 | 1.6 | 1.6 | 12.5 | 12.5 |
| DA2N (D) | 0.4 | 0.8 | 0.8 | 0.8 | 0.8 | 0.8 | 3.1 | 3.1 |
| Melittin | 2.5 | 2.5 | 5 | 5 | 5 | 5 | 1.2 | 1.2 |
| Gramicidin | >100 | >100 | >100 | >100 | >50 | >50 | 0.8 | 0.8 |

These results demonstrate that the DA2NE, DA2Na, K4D, L7A, K12A, K12P, V13A, A14D, A14K, M25A, DA2NEQ (D) and DA2N (D) peptides also have an antimicrobial activity on Gram-positive and Gram-negative bacteria, compared to the control peptides Scr L, Scr C, Melittin and Gramicidin.

1-12. (canceled)

13. A pharmaceutical composition or a medicament comprising:
at least one peptide with an anti-inflammatory, anti-angiogenic and/or antimicrobial activity having a sequence of 17 to 100 amino acids comprising the sequence A-L-X1-X2-T-L-X3-K-K-V-G-X4-X5-X6-G-K-A-cp (SEQ ID NO: 29), wherein:
X1 is a W or A residue;
X2 is a K or D residue;
X3 is an L or A residue;
X4 is a K, A or P residue;
X5 is a V or A residue;
X6 is an A, K or D residue;
φ is X7-L-N-A (SEQ ID NO: 30), X7-L-N-A-V-T-N-X8-A (SEQ ID NO: 31), X7-L-N-A-V-T-N-X8-A-N-Q-N-X9-X10 (SEQ ID NO: 32), or is absent; and wherein:
X7 is a V or P residue;
X8 is an M or A residue;
X9 represents no residue or is an E residue;
X10 represents no residue or is a Q residue; or
functional derivatives and pharmaceutically acceptable salts of said peptide, wherein the peptide is not a peptide of sequence SEQ ID NO: 2, or a nucleotide sequence coding for at least one of these peptides; and
one or a plurality of pharmaceutically acceptable excipients.

14. The pharmaceutical composition or medicament according to claim 13, wherein the peptide has a sequence of 29 to 100 amino acids comprising the sequence SEQ ID NO: 3, 4, 5, 6, 7 or 8.

15. A peptide with an anti-inflammatory activity and/or antimicrobial activity having a sequence of 17 to 100 amino acids comprising the sequence A-L-X1-X2-T-L-X3-K-K-V-G-X4-X5-X6-G-K-A-cp (SEQ ID NO: 29), wherein:
X1 is a W or A residue;
X2 is a K or D residue;
X3 is an L or A residue;
X4 is a K, A or P residue;
X5 is a V or A residue;
X6 is an A, K or D residue;
φ is X7-L-N-A (SEQ ID NO: 30), X7-L-N-A-V-T-N-X8-A (SEQ ID NO: 31), X7-L-N-A-V-T-N-X8-A-N-Q-N-X9-X10 (SEQ ID NO: 32), or is absent; and wherein:
X7 is a V or P residue;
X8 is an M or A residue;
X9 represents no residue or is an E residue;
X10 represents no residue or is a Q residue; and
functional derivatives and pharmaceutically acceptable salts of said peptide, wherein the peptide is not a peptide of sequence SEQ ID NO: 2, SEQ ID NO: 3 or SEQ ID NO: 4.

16. The peptide according to claim 15, wherein φ is X7-L-N-A-V-T-N-X8-A-N-Q-N-X9-X10 and X9 and X10 represent no residue.

17. The peptide according to claim 15 having a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 26, SEQ ID NO: 27, SEQ ID NO: 28 and functional derivatives and pharmaceutically acceptable salts of said peptide.

18. A method for preventing or treating an inflammatory disease or an angiogenesis-related disease or for attenuating or suppressing the immune reactions of the organism in a subject in need thereof, said method comprising administering to the subject an effective therapeutic dose of at least one peptide having a sequence of 17 to 100 amino acids comprising the sequence A-L-X1-X2-T-L-X3-K-K-V-G-X4-XS-X6-G-K-A-φ (SEQ ID NO: 29), wherein:
X1 is a W or A residue;
X2 is a K or D residue;
X3 is an L or A residue;
X4 is a K, A or P residue;
X5 is a V or A residue;
X6 is an A, K or D residue;
φ is X7-L-N-A (SEQ ID NO: 30), X7-L-N-A-V-T-N-X8-A (SEQ ID NO: 31), X7-L-N-A-V-T-N-X8-A-N-Q-N-X9-X10 (SEQ ID NO: 32), or is absent; and wherein:
X7 is a V or P residue;
X8 is an M or A residue;
X9 represents no residue or is an E residue;
X10 represents no residue or is a Q residue; or
functional derivatives and pharmaceutically acceptable salts of said peptide.

19. The method according to claim 18, wherein the peptide is not a peptide of SEQ ID NO: 2.

20. The method according to claim 18, wherein the method is for preventing or treating an inflammatory disease.

21. The method according to claim 20, wherein the peptide has a sequence of 29 to 100 amino acids comprising the sequence SEQ ID NO: 2, 3, 4, 5, 6, 7 or 8.

22. The method according to claim 20, wherein the inflammatory disease is selected from the group consisting of rosacea, psoriasis, eczema, contact dermatitis and impetigo.

23. The method according to claim 20, wherein the inflammatory disease is due to an infection.

24. The method according to claim 18, wherein the method is for preventing or treating an angiogenesis-related disease.

25. The method according to claim 24, wherein the peptide has a sequence of 29 to 100 amino acids comprising the sequence SEQ ID NO: 2, 3, 4, 5, 6, 7 or 8.

26. The method according to claim 24, wherein the angiogenesis-related disease is selected from the group consisting of cancers, retinopathy type ophthalmological disorders, atherosclerosis, arthrosis, and rheumatoid arthritis.


SEQUENCE LISTING (38 sequences)

SEQ ID NO: 1 - protein, 31 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
  SITE 18..18: Val or Pro
  SITE 25..25: Met or Ala
  SITE 30..30: no amino acid or Glu
  SITE 31..31: no amino acid or Gln
ALXXTLXKKVGXXXGKAXLNAVTNXANQNXX

SEQ ID NO: 2 - protein, 31 aa, Pachymedusa dacnicolor
ALWKTLLKKVGKVAGKAVLNAVTNMANQNEQ

SEQ ID NO: 3 - protein, 30 aa, Pachymedusa dacnicolor
ALWKTLLKKVGKVAGKAVLNAVTNMANQNE

SEQ ID NO: 4 - protein, 29 aa, Pachymedusa dacnicolor
ALWKTLLKKVGKVAGKAVLNAVTNMANQN

SEQ ID NO: 5 - protein, 31 aa, Artificial Sequence; identical to SEQ ID NO: 2
  Synthetic peptide
  MOD_RES 31..31: C-terminal amidation

SEQ ID NO: 6 - protein, 30 aa, Artificial Sequence; identical to SEQ ID NO: 3
  Synthetic peptide
  MOD_RES 30..30: C-terminal amidation

SEQ ID NO: 7 - protein, 29 aa, Artificial Sequence
  Synthetic peptide
  MOD_RES 29..29: C-terminal amidation
ALWKTLLKKVGKVAGKAVLNAVTNMANQN

SEQ ID NO: 8 - protein, 29 aa, Artificial Sequence
  Synthetic peptide
ALWKTLLKKVGKVKGKAVLNAVTNMANQN

SEQ ID NO: 9 - protein, 29 aa, Artificial Sequence
  Synthetic peptide
ALWKTLLKKVGKVAGKAPLNAVTNMANQN

SEQ ID NO: 10 - protein, 29 aa, Artificial Sequence
  Synthetic peptide
ALWKTLLKKVGKVDGKAVLNAVTNMANQN

SEQ ID NO: 11 - protein, 29 aa, Artificial Sequence
  Synthetic peptide
ALWKTLLKKVGAVAGKAVLNAVTNMANQN

SEQ ID NO: 12 - protein, 29 aa, Artificial Sequence
  Synthetic peptide
ALWKTLLKKVGPVAGKAVLNAVTNMANQN

SEQ ID NO: 13 - protein, 29 aa, Artificial Sequence
  Synthetic peptide
ALWDTLLKKVGKVAGKAVLNAVTNMANQN

SEQ ID NO: 14 - protein, 29 aa, Artificial Sequence
  Synthetic peptide
ALWKTLAKKVGKVAGKAVLNAVTNMANQN

SEQ ID NO: 15 - protein, 29 aa, Artificial Sequence
  Synthetic peptide
ALAKTLLKKVGKVAGKAVLNAVTNMANQN

SEQ ID NO: 16 - protein, 29 aa, Artificial Sequence
  Synthetic peptide
ALWKTLLKKVGKVAGKAVLNAVTNAANQN

SEQ ID NO: 17 - protein, 29 aa, Artificial Sequence
  Synthetic peptide
ALWKTLLKKVGKAAGKAVLNAVTNMANQN

SEQ ID NO: 18 - protein, 31 aa, Artificial Sequence; identical to SEQ ID NO: 2
  SITE 1..31: amino acid D
  Synthetic peptide

SEQ ID NO: 19 - protein, 29 aa, Artificial Sequence
  SITE 1..29: amino acid D
  Synthetic peptide
ALWKTLLKKVGKVAGKAVLNAVTNMANQN

SEQ ID NO: 20 - protein, 31 aa, Artificial Sequence
  SITE 1..31: amino acid D
  Synthetic peptide
QENQNAMNTVANLVAKGAVKGVKKLLTKWLA

SEQ ID NO: 21 - protein, 29 aa, Artificial Sequence
  SITE 1..29: amino acid D
  Synthetic peptide
NQNAMNTVANLVAKGAVKGVKKLLTKWLA

SEQ ID NO: 22 - DNA, 87 bp, Artificial Sequence
  Synthetic sequence
gcgctgtggaaaaccttactgaagaaagtgggcaaggttgcgggtaaagccgtgctgaacgcagttaccaatatggcgaaccagaat

SEQ ID NO: 23 - DNA, 90 bp, Artificial Sequence
  Synthetic sequence
gcgctgtggaaaaccttactgaagaaagtgggcaaggttgcgggtaaagccgtgctgaacgcagttaccaatatggcgaaccagaatgaa

SEQ ID NO: 24 - DNA, 93 bp, Artificial Sequence
  Synthetic sequence
gcgctgtggaaaaccttactgaagaaagtgggcaaggttgcgggtaaagccgtgctgaacgcagttaccaatatggcgaaccagaatgaacaa

SEQ ID NO: 25 - protein, 29 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
  SITE 18..18: Val or Pro
  SITE 25..25: Met or Ala
ALXXTLXKKVGXXXGKAXLNAVTNXANQN

SEQ ID NO: 26 - protein, 26 aa, Artificial Sequence
  Synthetic peptide
  MOD_RES 26..26: C-terminal amidation
ALWKTLLKKVGKVAGKAVLNAVTNMA

SEQ ID NO: 27 - protein, 21 aa, Artificial Sequence
  Synthetic peptide
  MOD_RES 21..21: C-terminal amidation
ALWKTLLKKVGKVAGKAVLNA

SEQ ID NO: 28 - protein, 17 aa, Artificial Sequence
  Synthetic peptide
  MOD_RES 17..17: C-terminal amidation
ALWKTLLKKVGKVAGKA

SEQ ID NO: 29 - protein, 31 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
  SITE 18..18: Pro, Val or vacant
  SITE 19..19: Leu or vacant, with the proviso that Xaa is Leu when Xaa in position 18 is Pro or Val, and that Xaa is vacant when Xaa in position 18 is vacant
  SITE 20..20: Asn or vacant, with the proviso that Xaa is Asn when Xaa in position 19 is Leu, and that Xaa is vacant when Xaa in position 19 is vacant
  SITE 21..21: Ala or vacant, with the proviso that Xaa is Ala when Xaa in position 20 is Asn, and that Xaa is vacant when Xaa in position 20 is vacant
  SITE 22..22: Val or vacant, with the proviso that Xaa is Val when Xaa in position 21 is Ala, and that Xaa is vacant when Xaa in position 21 is Ala or vacant
  SITE 23..23: Thr or vacant, with the proviso that Xaa is Thr when Xaa in position 22 is Val, and that Xaa is vacant when Xaa in position 22 is vacant
  SITE 24..24: Asn or vacant, with the proviso that Xaa is Asn when Xaa in position 23 is Thr, and that Xaa is vacant when Xaa in position 23 is vacant
  SITE 25..25: Ala, Met or vacant, with the proviso that Xaa is Ala or Met when Xaa in position 24 is Asn, and that Xaa is vacant when Xaa in position 24 is vacant
  SITE 26..26: Ala or vacant, with the proviso that Xaa is Ala when Xaa in position 25 is Ala or Met, and that Xaa is vacant when Xaa in position 25 is vacant
  SITE 27..27: Asn or vacant, with the proviso that Xaa is Asn when Xaa in position 26 is Ala, and that Xaa is vacant when Xaa in position 26 is Ala or vacant
  SITE 28..28: Gln or vacant, with the proviso that Xaa is Gln when Xaa in position 27 is Asn, and that Xaa is vacant when Xaa in position 27 is vacant
  SITE 29..29: Asn or vacant, with the proviso that Xaa is Asn when Xaa in position 28 is Gln, and that Xaa is vacant when Xaa in position 28 is vacant
  SITE 30..30: Gln, Glu or vacant, with the proviso that Xaa is Gln or Glu when Xaa in position 29 is Asn, and that Xaa is vacant when Xaa in position 29 is Asn or vacant
  SITE 31..31: Gln or vacant, with the proviso that Xaa is Gln when Xaa in position 30 is Glu, and that Xaa is vacant when Xaa in position 30 is Gln, Glu or vacant
ALXXTLXKKVGXXXGKAXXXXXXXXXXXXXX

SEQ ID NO: 30 - protein, 4 aa, Artificial Sequence
  SITE 1..1: Val or Pro
  Synthetic peptide
XLNA

SEQ ID NO: 31 - protein, 9 aa, Artificial Sequence
  SITE 1..1: Val or Pro
  Synthetic peptide
  SITE 8..8: Met or Ala
XLNAVTNXA

SEQ ID NO: 32 - protein, 14 aa, Artificial Sequence
  SITE 1..1: Val or Pro
  Synthetic peptide
  SITE 8..8: Met or Ala
  SITE 13..13: Glu or no amino acid
  SITE 14..14: Gln or no amino acid
XLNAVTNXANQNXX

SEQ ID NO: 33 - protein, 17 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
ALXXTLXKKVGXXXGKA

SEQ ID NO: 34 - protein, 21 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
  SITE 18..18: Val or Pro
ALXXTLXKKVGXXXGKAXLNA

SEQ ID NO: 35 - protein, 26 aa, Artificial Sequence
  Synthetic peptide
  SITE 3..3: Trp or Ala
  SITE 4..4: Lys or Asp
  SITE 7..7: Leu or Ala
  SITE 12..12: Lys, Ala or Pro
  SITE 13..13: Val or Ala
  SITE 14..14: Ala, Lys or Asp
  SITE 18..18: Val or Pro
  SITE 25..25: Met or Ala
ALXXTLXKKVGXXXGKAXLNAVTNXA

SEQ ID NO: 36 - protein, 75 aa, Pachymedusa dacnicolor
MALVKKSLFLVLFLGLVSLSICEEKRENEDEEEQEDDEQSEEKRALWKTLLKKVGKVAGKAVLNAVTNMANQNEQ

SEQ ID NO: 37 - protein, 31 aa, Artificial Sequence
  Synthetic peptide
NLGAKNQVAWEKKAATTKGNVNLKLLQMVAV

SEQ ID NO: 38 - protein, 29 aa, Artificial Sequence
  Synthetic peptide
KGNVLVKAAMLTKVLAKNKGVQNANALTW